(19) United States
(12) Patent Application Publication
(10) Pub. No.: US 2011/0065695 A1
(43) Pub. Date: Mar. 17, 2011

(54) USE OF AMINODIHYDROTHIAZINES FOR THE TREATMENT OR PREVENTION OF DIABETES

(76) Inventors: Jeremy Beauchamp, Riehen (CH); Agnès Bénardeau, Saint Louis (FR); Hans Hilpert, Muenchenstein (CH); Cristiano Mighliorini, Genf (CH); William Riboulet, Rixheim (FR); Haiyan Wang, Allschwil (CH)

(21) Appl. No.: 12/874,336

(22) Filed: Sep. 2, 2010

(30) Foreign Application Priority Data
Sep. 11, 2009 (EP) 09170126.8
Oct. 2, 2009 (EP) 09172068.0

(51) Int. Cl. A61K 31/541; C07D 417/12; C07D 279/06; C07D 495/04; A61K 31/54; A61P 3/10

(52) U.S. Cl. 514/227.2; 544/54

(57) ABSTRACT

This invention relates to compounds of formula I,

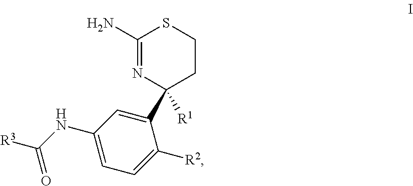

wherein R¹ to R³ are as defined herein, as well as pharmaceutically acceptable salts thereof, pharmaceutical compositions containing such compounds and methods for using such compounds for the treatment or prevention of diabetes, particularly type 2 diabetes.

### PRIORITY TO RELATED APPLICATION(S)

This application claims the benefit of European Patent Application No. 09170126.8, filed Sep. 11, 2009, and European Patent Application No. 09172068.0, filed Oct. 2, 2009 which are hereby incorporated by reference in its entirety.

### FIELD OF THE INVENTION

The present invention is concerned with the use of aminodihydrothiazine derivatives for the treatment or prevention of metabolic diseases such as preferably diabetes, particularly type 2 diabetes.

In particular, the present invention relates to the use of compounds of the general formula

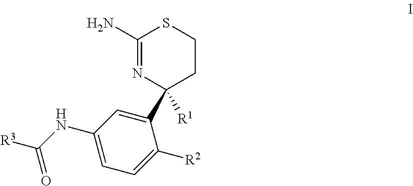

wherein R′, R², and R³ are as defined herein.

The compounds of formula I are selective inhibitors of BACE2.

### BACKGROUND OF THE INVENTION

Type 2 diabetes (T2D) is caused by insulin resistance and inadequate insulin secretion from pancreatic beta-cells leading to poor blood-glucose control and hyperglycemia (M Prentki & C J Nolan, “Islet beta-cell failure in type 2 diabetes.” J. Clin. Investig. 2006, 116(7), 1802-1812). Patients with T2D have an increased risk of microvascular and macrovascular disease and a range of related complications including diabetic nephropathy, retinopathy and cardiovascular disease. In 2000 an estimated 171 million people had the condition with the expectation that this figure will double by 2030 (S Wild, G Roglic, A Green, R. Sicree & H King, “Global prevalence of diabetes”, Diabetes Care 2004, 27(5), 1047-1053) making the disease a major healthcare problem. The rise in prevalence of T2D is associated with an increasingly sedentary lifestyle and high-energy food intake of the world's population (P Zimmet, K G M M Alberti & J Shaw, “Global and societal implications of the diabetes epidemic” Nature 2001, 414, 782-787).

Beta-cell failure and consequent dramatic decline in insulin secretion and hyperglycemia marks the onset of T2D (M Prentki & C J Nolan, “Islet beta-cell failure in type 2 diabetes.” J. Clin. Investig. 2006, 116(7), 1802-1812). Most current treatments do not prevent the loss of beta-cell mass characterising overt T2D. However, recent developments with GLP-1 analogues, gastrin and other agents show that preservation and proliferation of beta-cells is possible to achieve, leading to an improved glucose tolerance and slower progression to overt T2D (L L Baggio & D J Drucker, “Therapeutic approaches to preserve islet mass in type 2 diabetes”, Annu Rev. Med. 2006, 57, 265-281).

Tmem27 has been identified as a protein promoting beta-cell proliferation (P Akpinar, S Kuwajima, J Krützfeldt, M Stoffel, “Tmem27: A cleaved and shed plasma membrane protein that stimulates pancreatic β cell proliferation”, Cell Metab. 2005, 2, 385-397) and insulin secretion (K Fukui, Q Yang, Y Cao, N Takahashi et al., “The HNF-1 target Collectrin controls insulin exocytosis by SNARE complex formation”, Cell Metab. 2005, 2, 373-384). Tmem27 is a 42 kDa membrane glycoprotein which is constitutively shed from the surface of beta-cells, resulting from a degradation of the full-length cellular Tmem27. Overexpression of Tmem27 in a transgenic mouse increases beta-cell mass and improves glucose tolerance in a DIO model of diabetes [K Fukui, Q Yang, Y Cao, N Takahashi et al., “The HNF-1 target Collectrin controls insulin exocytosis by SNARE complex formation”, Cell Metab. 2005, 2, 373-384, P Akpinar, S Kuwajima, J Krützfeldt, M Stoffel, “Tmem27: A cleaved and shed plasma membrane protein that stimulates pancreatic β cell proliferation”, Cell Metab. 2005, 2, 385-397). Furthermore, siRNA knockout of Tmem27 in a rodent beta-cell proliferation assay (eg using INS1e cells) reduces the proliferation rate, indicating a role for Tmem27 in control of beta-cell mass.

In vitro, BACE2 cleaves a peptide based on the sequence of Tmem27. The closely related protease BACE1 does not cleave this peptide and selective inhibition of BACE1 alone does not enhance proliferation of beta-cells. BACE1 (BACE for beta-site APP-cleaving enzyme, also known as beta-secretase) has been implicated in the pathogenesis of Alzheimer disease and in the formation of myelin sheaths in peripheral nerve cells.

The close homolog BACE2 is a membrane-bound aspartyl protease and is colocalised with Tmem27 in rodent pancreatic beta-cells (G Finzi, F Franzi, C Placidi, F Acquati et al., “BACE2 is stored in secretory granules of mouse and rat pancreatic beta cells”, Ultrastruct Pathol. 2008, 32(6), 246-251). It is also known to be capable of degrading APP (1Hussain, D Powell, D Howlett, G Chapman et al., “ASP1 (BACE2) cleaves the amyloid precursor protein at the β-secretase site” Mol Cell Neurosci. 2000, 16, 609-619), IL-1R2 (P Kuhn, E Marjaux, A Imhof, B De Strooper et al., “Regulated intramembrane proteolysis of the interleukin-1 receptor II by alpha-, beta-, and gamma-secretase” J. Biol. Chem. 2007, 282(16), 11982-11995).

Inhibition of BACE2 is therefore proposed as a treatment for type 2 diabetes with the potential to preserve and restore beta-cell mass and stimulate insulin secretion in pre-diabetic and diabetic patients. It is therefore an object of the present invention to provide selective BACE2 inhibitors. Such compounds are useful as therapeutically active substances, particularly in the treatment and/or prevention of diseases which are associated with the inhibition of BACE2.

The compounds of the present invention exceed the compounds known in the art, inasmuch as they are strong and selective inhibitors of BACE2. They are expected to have an enhanced therapeutic potential compared to the compounds already known in the art and can be used for the treatment and prevention of diabetes, preferably type 2 diabetes, metabolic syndrome and a wide range of metabolic disorders.

### SUMMARY OF THE INVENTION

The present invention relates in part to a compound of formula Ia,

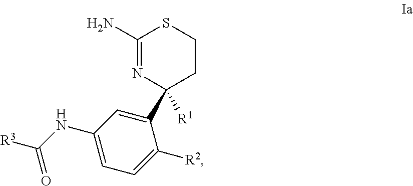

wherein
R¹ is ethyl;
R² is selected from the group consisting of C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

The present invention also relates to a pharmaceutical composition comprising a compound as described above and a pharmaceutically acceptable carrier and/or adjuvant.

The present invention further relates to a method for the treatment or prevention of diabetes, which method comprises administering, to a human being or animal in need thereof, a therapeutically active amount of a compound of formula I,

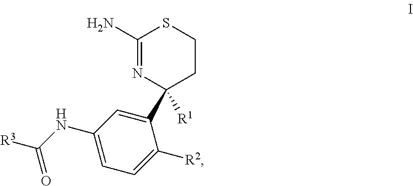

wherein
R¹ is C₁₋₇-alkyl or C₃₋₇-cycloalkyl;
R² is selected from the group consisting of hydrogen, C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

### BRIEF DESCRIPTION OF THE FIGURES

FIG. 1 is a graph showing results form an oral glucose tolerance test (oGTT) performed in 8.5 week old ZDF rats treated for 17 days with vehicle, Liraglutide or various amounts of the compound of Example 1. oGTT was performed on day 18.

FIG. 2 is a graph showing the glucose excursions during oGTT in 8.5 week old ZDF rats treated for 17 days with either vehicle, the compound of Example 1 or Liraglutide.

FIG. 3 is a graph illustrating the effect of chronic treatment with the compound of Example 1 on FBG measured before glucose challenge (fasting blood glucose after overnight fasting conditions).

FIG. 4 is a graph showing insulin levels during oGTT in 8.5 week old ZDF rats treated for 17 days with either vehicle, the compound of Example 1 or Liraglutide.

FIG. 5 is a graph showing the amounts of plasma Insulin AUC (0-120 minutes) during oGTT in 8.5 week old ZDF rats treated for 17 days with either vehicle, the compound of Example 1 or Liraglutide. AUC stands for Area Under the Curve. Y units are ng/ml*min.

FIG. 6 shows graphs illustrating the effect of treatment with either vehicle, the compound of Example l or Liraglutide on insulin resistance and insulin sensitivity as determined by the hepatic (HOMA) or whole body insulin resistance (MATSUDA) indexes as well as the β-cell sensitivity determined by HOMA-β index. The following calculations were made:

HOMA_IR index=(Fasting insulin (mU/ml)×FBG (mM)/22.5

ISI MATSUDA=1000/√(Go×Io×Gpriem×Ipriem), Priem=mean of glucose or insulin during OGTT.

HOMA-β cell=(20×FI)/(FBG−3.5).

Data were expressed as mean±SEM; (N=6 per group),
** in ISI MATSUDA means p<0.01 versus vehicle, ANOVA followed by Dunnett's Post Hoc test.

FIG. 7 is a graph showing in situ pancreatic insulin profiles (ng/ml) of 9 to 10 week old ZDF rats after treatment with either vehicle, the compound of Example 1 or Liraglutide. Last dose was administered 18 hours prior to pancreas perfusion (chronic effect).

FIG. 8 shows the results of immunoblotting of lysates of isolated human islets from two human donors that are not treated (−) or treated (+) with the compound of Example 1 for 72 h. Human pancreatic islets treated with the compound of Example 1 show preservation of full-length TMEM 27 and inhibit the autocatalytic activation of BACE2.

### DETAILED DESCRIPTION OF THE INVENTION

Unless otherwise indicated, the following definitions are set forth to illustrate and define the meaning and scope of the various terms used to describe the invention.

The term “halogen” refers to fluorine, chlorine, bromine and iodine, with fluorine, chlorine and bromine being preferred, and with fluorine and chlorine being more preferred.

The term “lower alkyl” or “C₁₋₇-alkyl”, alone or in combination, signifies a straight-chain or branched-chain alkyl group with 1 to 7 carbon atoms, preferably a straight or branched-chain alkyl group with 1 to 6 carbon atoms and particularly preferred a straight or branched-chain alkyl group with 1 to 4 carbon atoms. Examples of straight-chain and branched C₁₋₇ alkyl groups are methyl, ethyl, propyl, isopropyl, butyl, isobutyl, tert.-butyl, the isomeric pentyls, the isomeric hexyls and the isomeric heptyls, preferably methyl and ethyl and most preferred methyl.

The term “lower alkoxy” or “C₁₋₇-alkoxy” refers to the group R′—O—, wherein R′ is lower alkyl and the term “lower alkyl” has the previously given significance. Examples of lower alkoxy groups are methoxy, ethoxy, n-propoxy, isopropoxy, n-butoxy, isobutoxy, sec.-butoxy and tert.-butoxy, preferably methoxy and ethoxy.

The term “lower halogenalkyl” or “halogen-C₁₋₇-alkyl” refers to lower alkyl groups as defined above wherein at least one of the hydrogen atoms of the lower alkyl group is replaced by a halogen atom, preferably fluoro or chloro, most preferably fluoro. Among the preferred lower halogenalkyl groups are trifluoromethyl, difluoromethyl, trifluoroethyl, 2,2-difluoroethyl, fluoromethyl and chloromethyl, with trifluoromethyl or difluoromethyl being especially preferred.

The term “lower halogenalkoxy” or “halogen-C₁₋₇-alkoxy” refers to lower alkoxy groups as defined above wherein at least one of the hydrogen atoms of the lower alkoxy group is replaced by a halogen atom, preferably fluoro or chloro, most preferably fluoro. Among the preferred halogenated lower alkoxy groups are trifluoromethoxy, difluoromethoxy, fluormethoxy and chloromethoxy, with trifluoromethoxy being especially preferred.

The term “lower hydroxyalkyl” or “hydroxy-C₁₋₇-alkyl” refers to lower alkyl groups as defined above wherein at least one of the hydrogen atoms of the lower alkyl group is replaced by a hydroxy group. Among the preferred lower hydroxyalkyl groups are hydroxymethyl or hydroxyethyl.

The term “aryl” refers to an aromatic monocyclic or multicyclic ring system having 6 to 14 carbon atoms, preferably 6 to 10 carbon atoms. Preferred aryl groups are phenyl and naphthyl, with phenyl being most preferred.

The term “heteroaryl” refers to an aromatic or partly unsaturated 5- or 6-membered ring which comprises at least one heteroatom selected from nitrogen, oxygen and/or sulphur, and can in addition comprise one or three atoms selected from nitrogen, oxygen and/or sulphur, such as pyridyl, pyrazinyl, pyrimidinyl, pyridazinyl, 6-oxo-1,6-dihydropyridazinyl, 5-oxo-4,5-dihydropyrazinyl, pyrrolyl, furyl, thienyl, oxazolyl, isoxazolyl, oxadiazolyl, thiadiazolyl, tetrazolyl, pyrazolyl, imidazolyl, triazolyl and thiazolyl. The term “heteroaryl” further refers to bicyclic aromatic or partly unsaturated groups comprising two 5- or 6-membered rings, in which one or both rings can contain one, two or three atoms selected from nitrogen, oxygen or sulphur, such as quinolinyl, isoquinolinyl, cinnolinyl, pyrazolo[1,5-a]pyridyl, imidazo[1,2-a]pyridyl, thieno[2,3-c]pyridyl, quinoxalinyl, benzo[b]thienyl, benzothiazolyl, benzotriazolyl, indolyl, indazolyl and 3,4-dihydro-1H-isoquinolinyl. Preferred heteroaryl groups are thienyl, oxazolyl, thiazolyl, pyrazolyl, pyridyl, pyrimidinyl, pyrazinyl, isoquinolinyl, thieno[2,3-c]pyridyl and benzo[b]thienyl, with thienyl, oxazolyl, pyrazolyl, pyridyl, pyrimidinyl and pyrazinyl being more preferred and pyridyl being most preferred.

Compounds of formula I can form pharmaceutically acceptable salts. The term “pharmaceutically acceptable salts” refers to those salts which retain the biological effectiveness and properties of the free bases or free acids, which are not biologically or otherwise undesirable. Preferably, the pharmaceutically acceptable salts of the compounds of formula I are the acid addition salts with physiologically compatible mineral acids, such as hydrochloric acid, sulfuric acid, sulfurous acid or phosphoric acid; or with organic acids, such as methanesulfonic acid, ethanesulfonic acid, p-toluenesulfonic acid, formic acid, acetic acid, propionic acid, glycolic acid, pyruvic acid, oxylic acid, lactic acid, trifluoroacetic acid, citric acid, fumaric acid, maleic acid, malonic acid, tartaric acid, benzoic acid, cinnamic acid, mandelic acid, succinic acid or salicylic acid. Particularly preferred pharmaceutically acceptable salts of compounds of formula I are the acid addition salts such as the hydrochloride salts, the formate salts or trifluoroacetate salts.

The compounds of formula I can also be solvated, e.g., hydrated. The solvation can be effected in the course of the manufacturing process or can take place e.g. as a consequence of hygroscopic properties of an initially anhydrous compound of formula I (hydration). The term “pharmaceutically acceptable salts” also includes physiologically acceptable solvates.

“Isomers” are compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four non-identical substituents is termed a “chiral center”.

The present invention relates also to the use of a compound of the formula

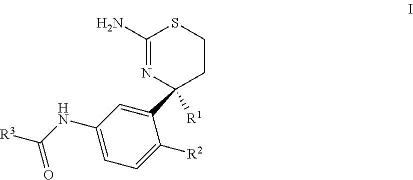

wherein
R¹ is C₁₋₇-alkyl or C₃₋₇-cycloalkyl;
R² is selected from the group consisting of hydrogen, C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof, for the preparation of a medicament for the treatment or prevention of metabolic disorders, preferably diabetes.

Preferably, the invention refers to the use as defined above of a compound of formula I, wherein R¹ is methyl or ethyl.

The use of a compound of formula I, wherein R² is selected from the group consisting of C₁₋₇-alkyl, halogen, cyano and C₁₋₇-alkoxy, is also preferred. More preferred is the use as defined above of a compound of formula I, wherein R² is halogen.

Further preferred is the use as defined above of a compound of formula I, wherein R³ is heteroaryl, said heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl. More preferably, R³ is heteroaryl selected from the group consisting of thienyl, oxazolyl, thiazolyl, pyrazolyl, pyridyl, pyrimidinyl, pyrazinyl, isoquinolinyl, thieno[2,3-c]pyridyl and benzo[b]thienyl, said heteroaryl being unsubstituted or substituted by one, two or three groups selected from the group consisting of C₁₋₇-alkyl, halogen, halogen-C₁₋₇-alkyl and phenyl.

Especially preferred is the use of a compound of formula I, which compound is 5-chloro-pyridine-2-carboxylic acid [3-((S)-2-amino-4-methyl-5,6-dihydro-4H-[1,3]thiazin-4-yl)-4-fluoro-phenyl]-amide (Compound J), or a pharmaceutically acceptable salt thereof, for the preparation of a medicament for the treatment or prevention of metabolic disorders, preferably diabetes.

Also preferred is the use as defined above of a compound of formula I, wherein R³ is phenyl, said phenyl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl.

The use of a compound of formula I as defined herein before for the preparation of a medicament for the treatment or prevention of type 2 diabetes is specifically preferred.

The invention also refers to a compound of the formula I,

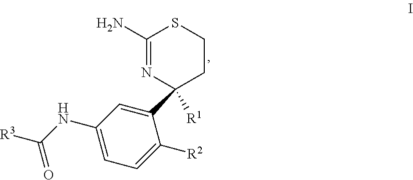

wherein
R¹ is C₁₋₇-alkyl or C₃₋₇-cycloalkyl;
R² is selected from the group consisting of hydrogen, C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

The compound may be used in the treatment or prevention of metabolic diseases, in particular in the treatment or prevention of diabetes, particularly type 2 diabetes.

Furthermore, the invention relates to a compound of formula I, wherein R¹ is methyl or ethyl.

The invention further relates to a compound of formula I, wherein R² is selected from the group consisting of C₁₋₇-alkyl, halogen, cyano and C₁₋₇-alkoxy, more particularly, wherein R² is halogen.

In particular, the invention refers to a compound of formula I, wherein R³ is heteroaryl, said heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl. More particularly, the invention relates to a compound of formula I for use in the treatment or prevention of metabolic diseases as defined above, wherein R³ is heteroaryl selected from the group consisting of thienyl, oxazolyl, thiazolyl, pyrazolyl, pyridyl, pyrimidinyl, pyrazinyl, isoquinolinyl, thieno[2,3-c]pyridyl and benzo[b]thienyl, said heteroaryl being unsubstituted or substituted by one, two or three groups selected from the group consisting of C₁₋₇-alkyl, halogen, halogen-C₁₋₇-alkyl and phenyl.

The invention further relates to a compound of formula I for use in the treatment or prevention of metabolic diseases as defined above, which compound is 5-chloro-pyridine-2-carboxylic acid [3-((S)-2-amino-4-methyl-5,6-dihydro-4H-[1,3]thiazin-4-yl)-4-fluoro-phenyl]-amide.

The invention also relates to a compound of formula I for use in the treatment or prevention of metabolic diseases as defined above, wherein R³ is phenyl, said phenyl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl.

Especially preferred is the compound of the formula I having the formula

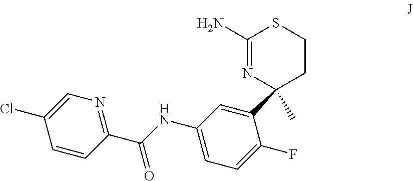

for use in the treatment or prevention of metabolic diseases, preferably for use in the treatment or prevention of diabetes, particularly type 2 diabetes.

Furthermore, the invention relates to new compounds of the formula I, wherein

R¹ is ethyl;
R² is selected from the group consisting of C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or pharmaceutically acceptable salts thereof.

Hereinafter, these will be referred to as compounds of formula Ia.

Preferred are compounds of formula Ia as defined above, wherein R² is halogen, with those compounds of formula Ia, wherein R² is fluoro, being most preferred.

Also preferred are compounds of formula Ia according to the invention, wherein R³ is heteroaryl, said heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl. More preferably, R³ is heteroaryl selected from the group consisting of thienyl, oxazolyl, thiazolyl, pyrazolyl, pyridyl, pyrimidinyl, pyrazinyl, isoquinolinyl, thieno[2,3-c]pyridyl and benzo[b]thienyl, said heteroaryl being unsubstituted or substituted by one, two or three groups selected from the group consisting of C₁₋₇-alkyl, halogen, halogen-C₁₋₇-alkyl and phenyl.

Further preferred compounds of formula Ia are those, wherein R³ is phenyl, said phenyl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl.

Particularly preferred compounds of formula Ia of the present invention are the following:
5-chloro-pyridine-2-carboxylic acid [3-((S)-2-amino-4-ethyl-5,6-dihydro-4H-[1,3]thiazin-4-yl)-4-fluoro-phenyl]-amide,
pyridine-2-carboxylic acid [3-((S)-2-amino-4-ethyl-5,6-dihydro-4H-[1,3]thiazin-4-yl)-4-fluoro-phenyl]-amide,
N-[3-((S)-2-amino-4-ethyl-5,6-dihydro-4H-[1,3]thiazin-4-yl)-4-fluoro-phenyl]-4-chloro-benzamide,
5-chloro-pyrazine-2-carboxylic acid [3-((S)-2-amino-4-ethyl-5,6-dihydro-4H-[1,3]thiazin-4-yl)-4-fluoro-phenyl]-amide,
5-chloro-pyrimidine-2-carboxylic acid [3-((S)-2-amino-4-ethyl-5,6-dihydro-4H-[1,3]thiazin-4-yl)-4-fluoro-phenyl]-amide,
3-trifluoromethyl-pyridine-2-carboxylic acid [3-((S)-2-amino-4-ethyl-5,6-dihydro-4H-[1,3]thiazin-4-yl)-4-fluoro-phenyl]-amide,
3-phenyl-pyridine-2-carboxylic acid [3-((S)-2-amino-4-ethyl-5,6-dihydro-4H-[1,3]thiazin-4-yl)-4-fluoro-phenyl]-amide,
4-chloro-pyridine-2-carboxylic acid [3-((S)-2-amino-4-ethyl-5,6-dihydro-4H-[1,3]thiazin-4-yl)-4-fluoro-phenyl]-amide,
6-methyl-pyridine-2-carboxylic acid [3-((S)-2-amino-4-ethyl-5,6-dihydro-4H-[1,3]thiazin-4-yl)-4-fluoro-phenyl]-amide,
3,6-dichloro-pyridine-2-carboxylic acid [3-((S)-2-amino-4-ethyl-5,6-dihydro-4H-[1,3]thiazin-4-yl)-4-fluoro-phenyl]-amide,
6-chloro-3-trifluoromethyl-pyridine-2-carboxylic acid [3-((S)-2-amino-4-ethyl-5,6-dihydro-4H-[1,3]thiazin-4-yl)-4-fluoro-phenyl]-amide,
isoquinoline-3-carboxylic acid [3-((S)-2-amino-4-ethyl-5,6-dihydro-4H-[1,3]thiazin-4-yl)-4-fluoro-phenyl]-amide,
thieno[2,3-c]pyridine-7-carboxylic acid [3-((S)-2-amino-4-ethyl-5,6-dihydro-4H-[1,3]thiazin-4-yl)-4-fluoro-phenyl]-amide,
benzo[1)]thiophene-2-carboxylic acid [3-((S)-2-amino-4-ethyl-5,6-dihydro-4H-[1,3]thiazin-4-yl)-4-fluoro-phenyl]-amide,
5-methyl-thiophene-2-carboxylic acid [3-((S)-2-amino-4-ethyl-5,6-dihydro-4H-[1,3]thiazin-4-yl)-4-fluoro-phenyl]-amide,
1-methyl-1H-pyrazole-3-carboxylic acid [3-((S)-2-amino-4-ethyl-5,6-dihydro-4H-[1,3]thiazin-4-yl)-4-fluoro-phenyl]-amide,
2-methyl-oxazole-4-carboxylic acid [3-((S)-2-amino-4-ethyl-5,6-dihydro-4H-[1,3]thiazin-4-yl)-4-fluoro-phenyl]-amide, and
2-methyl-thiazole-4-carboxylic acid [3-((S)-2-amino-4-ethyl-5,6-dihydro-4H-[1,3]thiazin-4-yl)-4-fluoro-phenyl]-amide,
   or pharmaceutically acceptable salts thereof.

The pharmaceutically acceptable salts of the compounds of formula Ia also individually constitute preferred compounds of the present invention.

Especially preferred are the salts of compounds of formula Ia with HCl, formic acid and trifluoroacetic acid (CF₃COOH), i.e. the chloride salts, the formate salts and trifluoroacetate salts. Most preferred are the salts of compounds of formula Ia with formic acid, i.e. the formate salts.

Within this group, the following salts are especially preferred:
5-chloro-pyridine-2-carboxylic acid [3-((S)-2-amino-4-ethyl-5,6-dihydro-4H-[1,3]thiazin-4-yl)-4-fluoro-phenyl]-amide; salt with formic acid,
pyridine-2-carboxylic acid [3-((S)-2-amino-4-ethyl-5,6-dihydro-4H-[1,3]thiazin-4-yl)-4-fluoro-phenyl]-amide; salt with formic acid,
N-[3-((S)-2-amino-4-ethyl-5,6-dihydro-4H-[1,3]thiazin-4-yl)-4-fluoro-phenyl]-4-chloro-benzamide; salt with formic acid,
5-chloro-pyrazine-2-carboxylic acid [3-((S)-2-amino-4-ethyl-5,6-dihydro-4H-[1,3]thiazin-4-yl)-4-fluoro-phenyl]-amide; salt with formic acid,
5-chloro-pyrimidine-2-carboxylic acid [3-((S)-2-amino-4-ethyl-5,6-dihydro-4H-[1,3]thiazin-4-yl)-4-fluoro-phenyl]-amide; salt with formic acid,
3-trifluoromethyl-pyridine-2-carboxylic acid [3-((S)-2-amino-4-ethyl-5,6-dihydro-4H-[1,3]thiazin-4-yl)-4-fluoro-phenyl]-amide; salt with formic acid,
3-phenyl-pyridine-2-carboxylic acid [3-((S)-2-amino-4-ethyl-5,6-dihydro-4H-[1,3]thiazin-4-yl)-4-fluoro-phenyl]-amide; salt with formic acid,
4-chloro-pyridine-2-carboxylic acid [3-((S)-2-amino-4-ethyl-5,6-dihydro-4H-[1,3]thiazin-4-yl)-4-fluoro-phenyl]-amide; salt with formic acid,
6-methyl-pyridine-2-carboxylic acid [3-((S)-2-amino-4-ethyl-5,6-dihydro-4H-[1,3]thiazin-4-yl)-4-fluoro-phenyl]-amide; salt with formic acid,
3,6-dichloro-pyridine-2-carboxylic acid [3-((S)-2-amino-4-ethyl-5,6-dihydro-4H-[1,3]thiazin-4-yl)-4-fluoro-phenyl]-amide; salt with formic acid,
6-chloro-3-trifluoromethyl-pyridine-2-carboxylic acid [3-((S)-2-amino-4-ethyl-5,6-dihydro-4H-[1,3]thiazin-4-yl)-4-fluoro-phenyl]-amide; salt with formic acid,
isoquinoline-3-carboxylic acid [3-((S)-2-amino-4-ethyl-5,6-dihydro-4H-[1,3]thiazin-4-yl)-4-fluoro-phenyl]-amide; salt with formic acid,
thieno[2,3-c]pyridine-7-carboxylic acid [3-((S)-2-amino-4-ethyl-5,6-dihydro-4H-[1,3]thiazin-4-yl)-4-fluoro-phenyl]-amide; salt with formic acid.
benzo[b]thiophene-2-carboxylic acid [3-((S)-2-amino-4-ethyl-5,6-dihydro-4H-[1,3]thiazin-4-yl)-4-fluoro-phenyl]-amide; salt with formic acid
5-methyl-thiophene-2-carboxylic acid [3-((S)-2-amino-4-ethyl-5,6-dihydro-4H-[1,3]thiazin-4-yl)-4-fluoro-phenyl]-amide; salt with formic acid
1-methyl-1H-pyrazole-3-carboxylic acid [3-((S)-2-amino-4-ethyl-5,6-dihydro-4H-[1,3]thiazin-4-yl)-4-fluoro-phenyl]-amide; salt with formic acid
2-methyl-oxazole-4-carboxylic acid [3-((S)-2-amino-4-ethyl-5,6-dihydro-4H-[1,3]thiazin-4-yl)-4-fluoro-phenyl]-amide; salt with formic acid, and
2-methyl-thiazole-4-carboxylic acid [3-((S)-2-amino-4-ethyl-5,6-dihydro-4H-[1,3]thiazin-4-yl)-4-fluoro-phenyl]-amide; salt with formic acid.

The skilled person in the art will recognize that the compounds of formula I can exist in tautomeric forms, e.g. in the following tautomeric form:

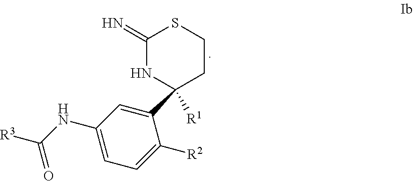

All tautomeric forms are encompassed in the present invention.

Compounds of formula I possess one asymmetric carbon atom and can exist in the form of optically pure enantiomers and mixtures of enantiomers such as, for example, racemates. The optically active forms can be obtained for example by resolution of the racemates, by asymmetric synthesis or asymmetric chromatography (chromatography with a chiral adsorbens or eluant). The invention embraces all of these forms.

The present invention is also concerned with the process for the manufacture of compounds of formula Ia as defined above, which process comprises

a) reacting an amine of the formula II

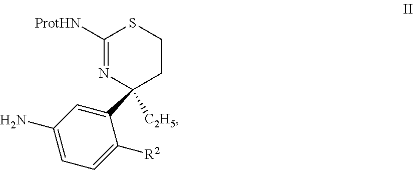

wherein R² is as defined in claim 1 and Prot is an amino protecting group, with a carboxylic acid of the formula III

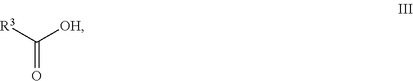

wherein R³ is as defined in claim 11, in the presence of a coupling reagent under basic conditions to obtain a compound of the formula IV

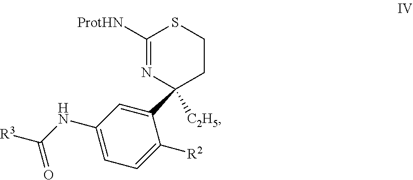

and deprotecting the compound of formula IV with the help of an acid to obtain the compound of formula I

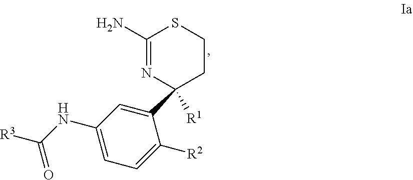

wherein R¹ to R³ are as defined in claim 11, and, if desired,
b) converting the compound obtained into a pharmaceutically acceptable salt.

The term “amino protecting group” refers to protecting groups such as Bz (benzoyl), Ac (acetyl), Trt (trityl), Boc (t-butyloxycarbonyl), CBz (benzyloxycarbonyl or Z), Fmoc (9-fluorenylmethoxycarbonyl), MBz (4-methoxyCBz), Poc (2-phenylpropyl(2)-oxycarbonyl) and Bpoc [(1-[1,1′-biphenyl]-4-yl-1-methylethoxy)carbonyl]. In particular, the amino protecting group is Boc (tert-butyloxycarbonyl).

Appropriate coupling agents are carbodiimides or uronium salts, such as for example N,N′-carbonyldiimidazole (CDI), N,N′-dicyclohexylcarbodiimide (DCC), N-(3-dimethylaminopropyl)-N′-ethyl-carbodiimide-hydrochloride (EDCI), O-(benzotriazol-1-yl)-N,N,N,N-tetramethyluronium tetrafluoroborate (TBTU) and 1-[bis(dimethylamino)methylene]-1H-1,2,3-triazolo[4,5-b]pyridinium-3-oxide hexafluorophosphate (HATU). The term “under basic conditions” means the presence of a base, preferably an alkylamine such as diisopropylethylamine (DIEA) or triethylamine (TEA), or a tertiary amine such as N-methylmorpholine or 4-(dimethylamino)-pyridine. The reaction is carried out in a suitable solvent such as for example N,N-dimethylformamide (DMF) or dimethylacetamide (DMAc), at temperatures between 0° C. and ambient temperature.

Preferred acids for the deprotection are sulfuric acid or hydrochloric acid, more preferably hydrochloric acid in a solvent such as an ether, preferably diethyl ether or 1,4-dioxane, or neat trifluoroacetic acid or formic acid, most preferably formic acid in a mixture of acetonitrile and water.

A more detailed description of the methods and procedures used for the preparation of compounds of formula I according to the present invention can be found in the examples.

As described herein before, the compounds of formula I or Ia of the present invention can be used as medicaments for the treatment of diseases which are associated with the inhibition of BACE2.

As described herein after, the compounds of formula I or Ia of the invention will be useful in preserving and restoring beta-cell function and stimulating insulin secretion in diabetic patients and in non-diabetic patients who have impaired glucose tolerance or who are in a pre-diabetic condition. They may be useful in preventing the onset or treating type 1 diabetes or in delaying or preventing a patient with type 2 diabetes from needing insulin therapy. The compounds of formula I are further useful to ameliorate hyperinsulinemia, which often occurs in diabetic or pre-diabetic patients and in reducing the risks associated with metabolic syndrome.

Thus, the expression ‘diseases which are associated with the inhibition of BACE2 activity’ means diseases such as metabolic and cardiovascular diseases, in particular diabetes, more particularly type 2 diabetes, gestational diabetes, impaired fasting glucose, impaired glucose tolerance, insulin resistance, pre-diabetes, metabolic syndrome, diabetes type 1, complications of diabetes including diabetic nephropathy, diabetic retinopathy and diabetic neuropathy, chronic kidney disease, dyslipidemia, atherosclerosis, myocardial infarction, hypertension and further metabolic and cardiovascular disorders. In a preferable aspect, the expression ‘diseases which are associated with the inhibition of BACE2 activity’ relates to diabetes, particularly type II diabetes, impaired glucose tolerance, pre-diabetes, metabolic syndrome. More preferably, the expression ‘diseases which are associated with the inhibition of BACE2 activity’ relates to diabetes, most preferably type 2 diabetes.

The invention also relates to pharmaceutical compositions comprising a compound of formula Ia as defined above and a pharmaceutically acceptable carrier and/or adjuvant. More specifically, the invention relates to pharmaceutical compositions useful for the treatment of diseases which are associated with the inhibition of BACE2 activity.

Further, the invention relates to compounds of formula Ia as defined above for use as medicaments, particularly as medicaments for the treatment or prevention of diseases which are associated with the inhibition of BACE2 activity. Especially preferred are compounds of formula I for use in diabetes, particularly type 2 diabetes.

The compounds of formula I or Ia and their pharmaceutically acceptable salts can be used as medicaments, e.g., in the form of pharmaceutical preparations for enteral, parenteral or topical administration. They can be administered, for example, perorally, e.g., in the form of tablets, coated tablets, dragées, hard and soft gelatine capsules, solutions, emulsions or suspensions, rectally, e.g., in the form of suppositories, parenterally, e.g., in the form of injection solutions or suspensions or infusion solutions, or topically, e.g., in the form of ointments, creams or oils. Oral administration is preferred.

The production of the pharmaceutical preparations can be effected in a manner which will be familiar to any person skilled in the art by bringing the described compounds of formula I and their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the compound could be administered with one or several daily dosage units, e.g., in 1 to 3 dosage units.

The pharmaceutical preparations conveniently contain about 1-500 mg, preferably 1-100 mg, of a compound of formula I.

In another aspect, the invention relates to a method for the treatment or prevention of diseases which are associated with the inhibition of BACE2 activity, preferably diabetes, particularly type 2 diabetes, which method comprises administering, to a human being or animal in need thereof, a therapeutically active amount of a compound of formula I,

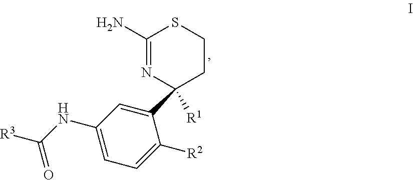

wherein
R¹ is C₁₋₇-alkyl or C₃₋₇-cycloalkyl;
R² is selected from the group consisting of hydrogen, C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

The effects of a compound of formula I on metabolic parameters such as blood glucose, plasma insulin, insulin resistance and insulin sensitivity were evaluated in a long-term study with 6 week old Zucker Diabetic Fatty (ZDF) rats treated for 4 weeks. The long-acting GLP-1 analog Liraglutide (NN2211, CAS Registry No. 204656-20-2) was used as positive control. Liraglutide has been launched under the tradename Victoza in the UK and Germany for the treatment of type 2 diabetes. After 3 weeks of treatment an oral Glucose Tolerance Test (oGTT) was performed on overnight fasted rats. After 3 to 4 weeks of treatment and after anesthesia, ZDF rats (2/3 per day) underwent pancreas surgery and in-situ perfusion with low/high glucose medium. The results of this study are discussed in Example 21.

In summary, the compound of formula I (Example 1) reduced post-challenged glucose levels of ZDF rats after 17 days of oral treatment and thus improves after chronic treatment pancreas function as measured by improvement of glucose tolerance. The compound of formula I further increased insulin levels (at peak and up to 60 minutes post glucose challenge) of ZDF rats after 17 days of oral treatment and 18 h after last dosing (chronic effect). Chronic treatment with a compound of formula I did not impact on hepatic (HOMA) or peripheral (MATSUDA) insulin resistance indexes. In contrast, treatment with a compound of formula I improved HOMA β-cell index. The treatment with a compound of formula I reduced basal pancreatic insulin secretion and thus normalized the pancreatic insulin secretion profile to that of 6 weeks old non-diabetic ZDF rats. Compounds of formula I may therefore be useful for protecting pancreas function and the prevention of hyperinsulinemia.

### EXAMPLES

The following examples serve to illustrate the present invention in more detail. They are, however, not intended to limit its scope in any manner.

### ABBREVIATIONS

DIEA=diisopropylethylamine, DMF=N,N-dimethylformamide, HATU=1-[bis(dimethylamino)methylene]-1H-1,2,3-triazolo[4,5-b]pyridinium-3-oxide hexafluorophosphate, HPLC=high performance liquid chromatography, LDA=lithium diisopropylamide, MS=mass spectrum and THF=tetrahydrofuran.

### Example 1

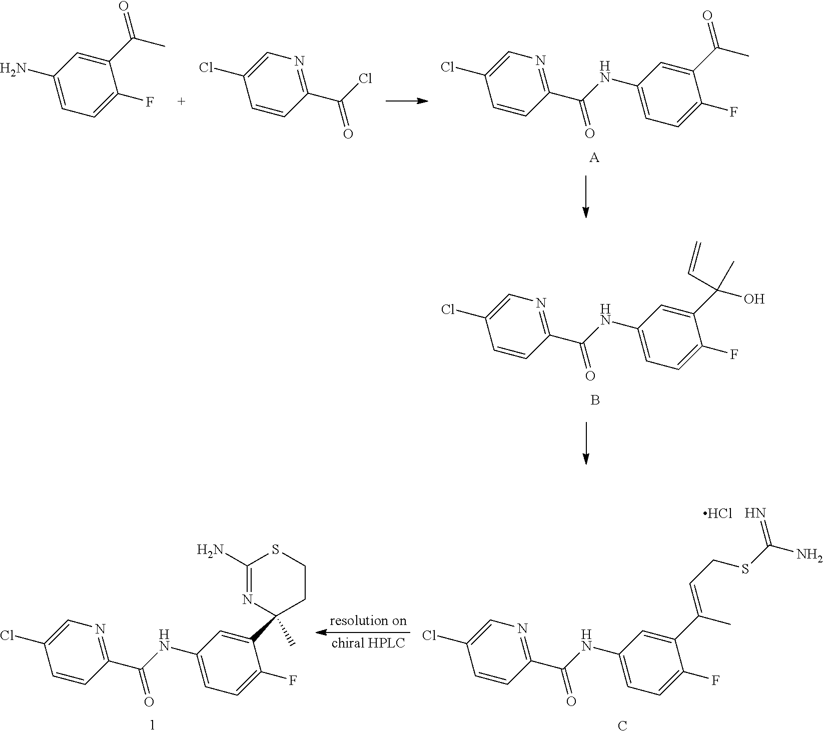

### Synthesis of the intermediate 5-chloro-pyridine-2-carboxylic acid β-acetyl-4-fluoro-phenyl)-amide (A)

To a solution of 5-chloro-pyridine-2-carbonyl chloride (30.5 g, preparation described in H. G. Brunner, EP353187, 1990) in THF (750 ml) was added subsequently 1-(5-amino-2-fluoro-phenyl)-ethanone (25.3 g, preparation described in M. Q. Zhang et al., J. Heterocyclic Chem. 28, 673, 1991) and NEt3 (18.4 g) keeping the temperature between 20-30° C. The suspension was stirred at 22° C. for 2 h and evaporated. The residue was partitioned between ethyl acetate and saturated aqueous NaHCO₃, the organic layer was washed with water, dried and evaporated. The residue was triturated with pentane, filtered and the residue dried to give the title compound (48.0 g, 99%) as a pale brown solid. MS (ESI): m/z=293.0 [M+1]⁺.

### Synthesis of the intermediate 5-chloro-pyridine-2-carboxylic acid [4-fluoro-3-(1-hydroxy-1-methyl-allyl)-phenyl]-amide (B)

To a suspension of 5-chloro-pyridine-2-carboxylic acid β-acetyl-4-fluoro-phenyl)-amide (47.7 g) in THF (850 ml) and diethyl ether (850 ml) was added at −78° C. vinylmagnesium chloride (1.7 M in THF, 240 ml) keeping the temperature below −60° C. The mixture was stirred at −60° C. for 1 h and at −20° C. for 3 h and quenched with saturated aqueous NH4Cl (1500 ml). The mixture was diluted with ethyl acetate (250 ml), the layers were separated and the aqueous layer was extracted again with ethyl acetate. The combined organic layers were washed with saturated aqueous NaHCO3 (600 ml) and brine (600 ml), dried and evaporated. The residue was dissolved in boiling ethyl acetate (80 ml), and evaporated again until a thick suspension was obtained. The suspension was diluted with a mixture of pentane/diethyl ether (3:1, 20 ml) and neat pentane (50 ml), filtered and the residue dried to give the title compound (40.0 g, 77%) as a pale yellow solid. MS (ESI): m/z=319.1 [M−1]⁻.

### Synthesis of the intermediate 5-chloro-pyridine-2-carboxylic acid [3-((E)-3-carbamimidoylsulfanyl-1-methyl-propenyl)-4-fluoro-phenyl]-amide; salt with HCl (C)

A solution of thiourea (11.11 g) and 5-chloro-pyridine-2-carboxylic acid [4-fluoro-3-(1-hydroxy-1-methyl-allyl)-phenyl]-amide (46.8 g) in a solution of HCl in acetic acid (1M, 260 ml) was stirred at 22° C. for 30 min and at 40° C. for 3 h. The mixture was evaporated, the residue was codistilled with toluene and triturated with ethyl ether (600 ml). The suspension was filtered and the residue dried to give the title compound (54.2 g, 90%) as a pale brown solid. MS (ESI): m/z=379.2 [M+1]⁺.

### Synthesis of 5-chloro-pyridine-2-carboxylic acid [3-((S)-2-amino-4-methyl-5,6-dihydro-4H-[1,3]thiazin-4-yl)-4-fluoro-phenyl]-amide (Compound J)

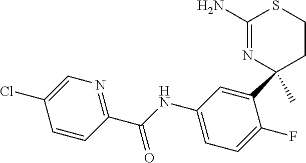

To a brown solution of 5-chloro-pyridine-2-carboxylic acid [3-((E)-3-carbamimidoylsulfanyl-1-methyl-propenyl)-4-fluoro-phenyl]-amide; salt with HCl (54.8 g) in trifluoroacetic acid (275 ml) was added at 0° C. trifluoromethanesulfonic acid (31.5 ml) and stirring was continued at 22° C. for 3 h. The mixture was evaporated and the residue partitioned between saturated aqueous Na2CO3 and ethyl acetate. The aqueous layer was extracted twice with ethyl acetate and the combined organic layers were washed with brine. Since the product precipitated during the washing procedure already, the suspension was filtered to give the racemic title product as an off-white solid (4.81 g, 10%). The layers of the filtrate were separated, the organic layer was dried and evaporated to a volume of approximately 400 ml and filtered. The residue was washed with ethyl acetate and diethyl ether and dried to give a second portion of the racemic title compound as an off-white solid (22.6 g, 45%). MS (ESI): m/z=379.2 [M+1]

The racemate was resolved on a chiral HPLC column (Chiralpak AD, 20 uM, 250×110 mm) using acetonitrile/1-propanol (85:15) in 8 batches to give 5-chloro-pyridine-2-carboxylic acid [3-((R)-2-amino-4-methyl-5,6-dihydro-4H-[1,3]thiazin-4-yl)-4-fluoro-phenyl]-amide (12.8 g) as the faster eluting product and 5-chloro-pyridine-2-carboxylic acid [3-((S)-2-amino-4-methyl-5,6-dihydro-4H-[1,3]thiazin-4-yl)-4-fluoro-phenyl]-amide (12.0 g) as the slower eluting product.

### Example 2-19

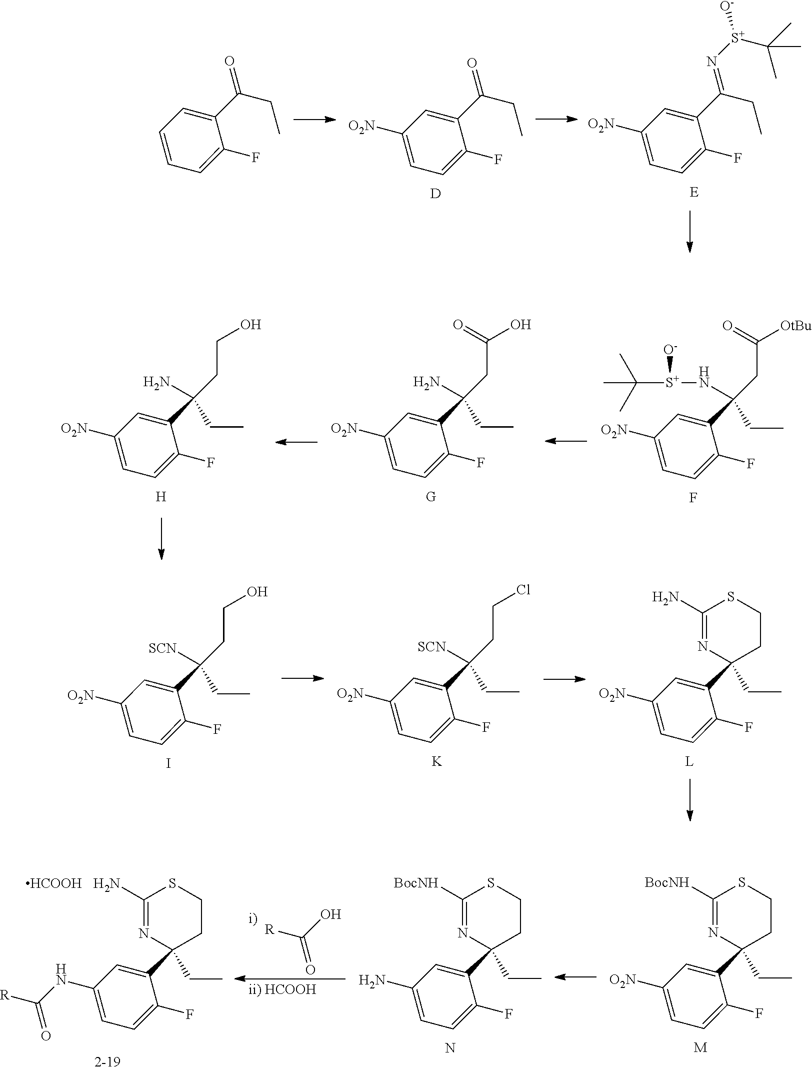

### Synthesis of the intermediate 1-(2-fluoro-5-nitrophenyl)propan-1-one (D)

1-(2-Fluorophenyl)propan-1-one (54 g, 355 mmol) was added dropwise to sulfuric acid (180 mL) at −20° C., then fuming nitric acid (27 mL) was added to the mixture at such a rate that the temperature never exceeded −15° C. The mixture was stirred for 10 minutes, then poured into ice, extracted with ethyl acetate, washed with H₂O, aqueous NaHCO₃ and brine, dried (Na₂SO₄) and evaporated. The crude material was chromatographed over silica (pentane/ethyl acetate, 10:1) to give the title product (40 g, 58%). MS (ESI): m/z=198.0 [M+1]⁺.

### Synthesis of the intermediate (R)-2-methyl-propane-2-sulfinic acid [1-(2-fluoro-5-nitro-phenyl)-prop-(E)-ylidene]-amide (E)

1-(2-Fluoro-5-nitrophenyl)propan-1-one (41.5 g, 211 mmol) and (R)-(+)-tert-butylsulfinamide (51.0 g, 421 mmol) were dissolved in THF (250 mL), then added titanium(IV) ethoxide (154 g, 675 mmol) at room temperature, the mixture was stirred at 70° C. for 3 hours and cooled to room temperature. The mixture was treated with brine (400 ml), the suspension was stirred for 10 min and filtered over dicalite. The layers were separated, the aqueous layer was extracted with ethyl acetate, the combined organic layers were washed with water, dried and evaporated. The residue was chromatographed on silica using pentane/ethyl:acetate (5:1) to give the title product (50 g, 78%). MS (ESI): m/z=301.0 [M+1]⁺.

### Synthesis of the intermediate (S)-tert-butyl 3-((R)-1,1-dimethylethylsulfinamido)-3-(2-fluoro-5-nitrophenyl)pentanoate (F)

A solution of tBuOAc (40.0 g, 351 mmol) in THF (200 mL) was added to a solution of LDA (2M 200 mL) at −78° C., the mixture was stirred at the same temperature for 30 minutes, then triisopropoxytitanium (IV) chloride (92.0 g, 353 mmol) in THF (200 mL) was added to the mixture. Half an hour later, (R)-2-methyl-propane-2-sulfinic acid [1-(2-fluoro-5-nitro-phenyl)-prop-(E)-ylidene]-amide (30.0 g, 100 mmol) was added to the mixture, the mixture was stirred at −78° C. for 1 hour and then poured into aqueous NH₄Cl solution with ice-water bath cooling. The mixture was diluted with ethyl acetate, filtrated, the organic layer was washed with brine, dried over Na₂SO₄, and purified by chromatography (pentane/ethyl acetate, 3:1) to give the title compound (20.9 g, 61%). MS (ESI): m/z=417.0 [M+1]⁺.

### Synthesis of the intermediate (S)-3-amino-3-(2-fluoro-5-nitrophenyl)pentanoic acid (G)

(S)-Tert-butyl 3-((R)-1,1-dimethylethylsulfinamido)-3-(2-fluoro-5-nitrophenyl)pentanoate (20.9 g, 50.0 mmol) was dissolved in HCl (300 mL, 4 M in 1,4-dioxane), then the mixture was stirred for 15 hours at 90° C. The mixture was cooled to room temperature and concentrated under reduced pressure. The brown oil was triturated with ether to give the title product (10.0 g, 66.0%). MS (ESI): m/z=257.0 [M+1]⁺.

### Synthesis of the intermediate (S)-3-amino-3-(2-fluoro-5-nitrophenyl)pentan-1-ol (H)

(S)-3-Amino-3-(2-fluoro-5-nitrophenyl)pentanoic acid (10.0 g, 39.0 mmol) was suspended in THF (100 mL) and treated dropwise with borane (200 mL, 1M in THF). The mixture was stirred at room temperature for 30 hours and then poured into ice-water. The mixture was basified to pH=9 with 4 N sodium hydroxide aqueous solution, extracted with ethyl acetate, the organic layer was washed with brine, dried over Na₂SO₄ and concentrated to give the title product (5.0 g, 60%). MS (ESI): m/z=243.0 [M+1]⁺.

### Synthesis of the intermediate (S)-3-(2-fluoro-5-nitro-phenyl)-3-isothiocyanato-pentan-1-ol (I)

(S)-3-Amino-3-(2-fluoro-5-nitrophenyl)pentan-1-ol (5.0 g, 21.0 mmol) was suspended in a mixture of toluene (30 mL) and water (30 mL). To the suspension was added potassium carbonate (8.0 g, 58 mmol) followed by thiophosgene (2.85 g, 25 mmol) under ice-water bath cooling. The mixture was stirred for half hour, diluted with ethyl acetate (100 ml) and water (50 mL) and the mixture was filtrated. The organic layer was washed with brine, dried over Na₂SO₄ and concentrated at reduced pressure to give the crude title compound (5.0 g) as dark oil which was used directly in the next step.

### Synthesis of the intermediate 2-((S)-3-chloro-1-ethyl-1-isothiocyanato-propyl)-1-fluoro-4-nitro-benzene (K)

To a solution of (S)-3-(2-fluoro-5-nitro-phenyl)-3-isothiocyanato-pentan-1-ol (5.0 g, crude) in toluene (50 mL) was added thionyl chloride (5.0 mL, 70 mmol) and DMF (0.5 mL) and the mixture was heated at 80° C. for 3 hours. The mixture was cooled to 22° C., poured into ice-water and extracted with ethyl acetate. The organic layer was washed with brine, dried over Na₂SO₄ and purified by chromatography (pentane/ethyl acetate, 20:1) to give the title compound (4.0 g, 64%).

### Synthesis of the intermediate (S)-4-ethyl-4-(2-fluoro-5-nitrophenyl)-5,6-dihydro-4H-[1,3]thiazin-2-ylamine (L)

To a solution of 2-((S)-3-chloro-1-ethyl-1-isothiocyanato-propyl)-1-fluoro-4-nitro-benzene (4.0 g, 13 mmol) in THF (40 ml) was added ammonia in water (26 mL, 25-28%) under ice-water bath cooling and the mixture was stirred for 6 hours at room temperature. The mixture was diluted with water and ethyl acetate, the organic layer was washed with brine, dried over Na₂SO₄ and concentrated at reduced pressure to give the crude title product (3.0 g, 80%).

### Synthesis of the intermediate [(S)-4-ethyl-4-(2-fluoro-5-nitro-phenyl)-5,6-dihydro-4H-[1,3]thiazin-2-yl]-carbamic acid tert-butyl ester (M)

To a solution of (S)-4-ethyl-4-(2-fluoro-5-nitrophenyl)-5,6-dihydro-4H-[1,3]thiazin-2-ylamine (3.0 g, 10.6 mmol) in dichloromethane (50 mL) was added Et₃N (3.2 g, 31.8 mmol) and Boc₂O (2.78 g, 12.7 mmol) and stirring was continued at 22° C. for 10 h. The mixture was evaporated, the residue partitioned between ethyl acetate and water, the organic layer was dried over Na₂SO₄, evaporated and purified by chromatography to give the title product (3.5 g, 88%). MS (ESI): m/z=384.0 [M+1]⁺.

### Synthesis of the intermediate [(S)-4-(5-amino-2-fluoro-phenyl)-4-ethyl-5,6-dihydro-4H-[1,3]thiazin-2-yl]-carbamic acid tert-butyl ester (N)

To a solution of [(S)-4-ethyl-4-(2-fluoro-5-nitro-phenyl)-5,6-dihydro-4H-[1,3]thiazin-2-yl]-carbamic acid tert-butyl ester (3.4 g, 8.9 mmol) in methanol (50 mL) was added Pd/C (5.0 g, 10%) and the mixture was hydrogenated at 30 Psi for 2 h. The catalyst was removed by filtration, the filtrate was evaporated and the residue was purified by column chromatography (pentane/ethyl acetate, 3:1) to give the pure title product (2.3 g, 74%). MS (ESI): m/z=354.0 [M+1]⁺.

### Coupling of [(S)-4-(5-amino-2-fluoro-phenyl)-4-ethyl-5,6-dihydro-4H-[1,3]thiazin-2-yl]-carbamic acid tert-butyl ester and a carbonic acid

### General Procedure

To a solution of [(S)-4-(5-amino-2-fluoro-phenyl)-4-ethyl-5,6-dihydro-4H-[1,3]thiazin-2-yl]-carbamic acid tert-butyl ester (0.11 mmole) in DMF (0.8 ml) was added subsequently HATU (0.14 mmole), the carbonic acid (0.13 mmole) and DIEA (0.44 mmole) and stirring was continued at 22° C. for 2 h. The mixture was acidified with formic acid and purified on prep. RP-18 HPLC using a gradient of acetonitrile and water (containing 0.1% of formic acid). Fractions containing the t-butyloxycarbonyl protected intermediate were evaporated, the residue was dissolved in a mixture of H₂O/CH₃CN/HCOOH (1:1:0.1, 2.0 ml) and stirred at 50° C. for 2 h. The mixture was evaporated to give the pure amides as the formic acid salt.

### Example 2

### 5-Chloro-pyridine-2-carboxylic acid [3-((S)-2-amino-4-ethyl-5,6-dihydro-4H-[1,3]thiazin-4-yl)-4-fluoro-phenyl]-amide; salt with formic acid

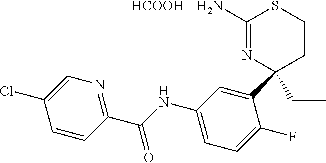

The coupling of [(S)-4-(5-amino-2-fluoro-phenyl)-4-ethyl-5,6-dihydro-4H-[1,3]thiazin-2-yl]-carbamic acid tert-butyl ester and 5-chloro-pyridine-2-carboxylic acid followed by deprotection of the intermediate yielded the title compound (24 mg) as a colourless solid. MS (ESI): m/z=393.2 [M+H]⁺.

### Example 3

### Pyridine-2-carboxylic acid [3-((S)-2-amino-4-ethyl-5,6-dihydro-4H-[1,3]thiazin-4-yl)-4-fluoro-phenyl]-amide; salt with formic acid

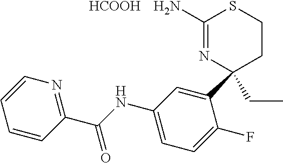

The coupling of [(S)-4-(5-amino-2-fluoro-phenyl)-4-ethyl-5,6-dihydro-4H-[1,3]thiazin-2-yl]-carbamic acid tert-butyl ester and pyridine-2-carboxylic acid followed by deprotection of the intermediate yielded the title compound (31 mg) as a pale yellow solid. MS (ESI): m/z=359.3 [M+H]⁺.

### Example 4

### N-[3-((S)-2-Amino-4-ethyl-5,6-dihydro-4H-[1,3]thiazin-4-yl)-4-fluoro-phenyl]-4-chloro-benzamide; salt with formic acid

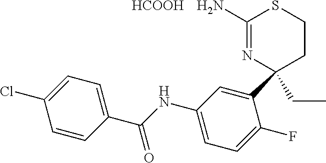

The coupling of [(S)-4-(5-amino-2-fluoro-phenyl)-4-ethyl-5,6-dihydro-4H-[1,3]thiazin-2-yl]-carbamic acid tert-butyl ester and 4-chloro-benzoic acid followed by deprotection of the intermediate yielded the title compound (27 mg) as a colorless solid. MS (ESI): m/z=392.2 [M+H]⁺.

### Example 5

### 5-Chloro-pyrazine-2-carboxylic acid [3-((S)-2-amino-4-ethyl-5,6-dihydro-4H-[1,3]thiazin-4-yl)-4-fluoro-phenyl]-amide; salt with formic acid

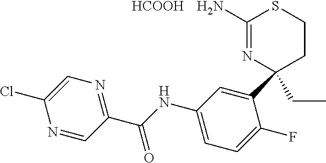

The coupling of [(S)-4-(5-amino-2-fluoro-phenyl)-4-ethyl-5,6-dihydro-4H-[1,3]thiazin-2-yl]-carbamic acid tert-butyl ester and 5-chloro-pyrazine-2-carboxylic acid followed by deprotection of the intermediate yielded the title compound (13 mg) as a colorless solid. MS (ESI): m/z=394.1 [M+H]⁺.

### Example 6

### 5-Chloro-pyrimidine-2-carboxylic acid [3-((S)-2-amino-4-ethyl-5,6-dihydro-4H-[1,3]thiazin-4-yl)-4-fluoro-phenyl]-amide; salt with formic acid

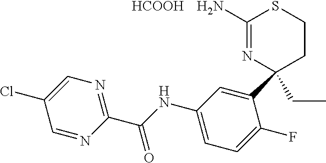

The coupling of [(S)-4-(5-amino-2-fluoro-phenyl)-4-ethyl-5,6-dihydro-4H-[1,3]thiazin-2-yl]-carbamic acid tert-butyl ester and 5-chloro-pyrimidine-2-carboxylic acid followed by deprotection of the intermediate yielded the title compound (25 mg) as a pale yellow solid. MS (ESI): m/z=394.1 [M+H]⁺.

### Example 7

### 3-Trifluoromethyl-pyridine-2-carboxylic acid [3-((S)-2-amino-4-ethyl-5,6-dihydro-4H-[1,3]thiazin-4-yl)-4-fluoro-phenyl]-amide; salt with formic acid

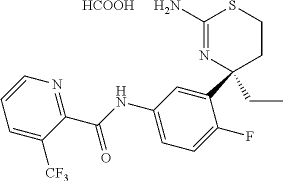

The coupling of [(S)-4-(5-amino-2-fluoro-phenyl)-4-ethyl-5,6-dihydro-4H-[1,3]thiazin-2-yl]-carbamic acid tert-butyl ester and 3-trifluoromethyl-pyridine-2-carboxylic acid followed by deprotection of the intermediate yielded the title compound (36 mg) as a colorless solid. MS (ESI): m/z=427.2 [M+H]⁺.

### Example 8

### 3-Phenyl-pyridine-2-carboxylic acid [3-((S)-2-amino-4-ethyl-5,6-dihydro-4H-[1,3]thiazin-4-yl)-4-fluoro-phenyl]-amide; salt with formic acid

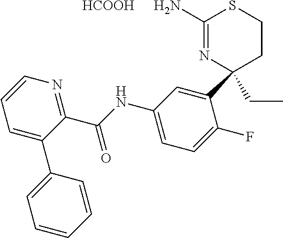

The coupling of [(S)-4-(5-amino-2-fluoro-phenyl)-4-ethyl-5,6-dihydro-4H-[1,3]thiazin-2-yl]-carbamic acid tert-butyl ester and 3-phenyl-pyridine-2-carboxylic acid followed by deprotection of the intermediate yielded the title compound (38 mg) as a colorless solid. MS (ESI): m/z=435.3 [M+H]⁺.

### Example 9

### 4-Chloro-pyridine-2-carboxylic acid [3-((S)-2-amino-4-ethyl-5,6-dihydro-4H-[1,3]thiazin-4-yl)-4-fluoro-phenyl]-amide; salt with formic acid

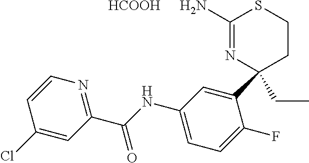

The coupling of [(S)-4-(5-amino-2-fluoro-phenyl)-4-ethyl-5,6-dihydro-4H-[1,3]thiazin-2-yl]-carbamic acid tert-butyl ester and 4-chloro-pyridine-2-carboxylic acid followed by deprotection of the intermediate yielded the title compound (31 mg) as a colorless oil. MS (ESI): m/z=393.2 [M+H]⁺.

### Example 10

### 6-Methyl-pyridine-2-carboxylic acid [3-((S)-2-amino-4-ethyl-5,6-dihydro-4H-[1,3]thiazin-4-yl)-4-fluoro-phenyl]-amide; salt with formic acid

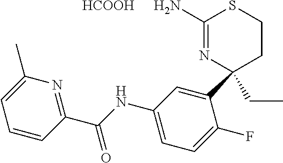

The coupling of [(S)-4-(5-amino-2-fluoro-phenyl)-4-ethyl-5,6-dihydro-4H-[1,3]thiazin-2-yl]-carbamic acid tert-butyl ester and 6-methyl-pyridine-2-carboxylic acid followed by deprotection of the intermediate yielded the title compound (28 mg) as a colorless oil. MS (ESI): m/z=373.1 [M+H]⁺.

### Example 11

### 3,6-Dichloro-pyridine-2-carboxylic acid [3-((S)-2-amino-4-ethyl-5,6-dihydro-4H-[1,3]thiazin-4-yl)-4-fluoro-phenyl]-amide; salt with formic acid

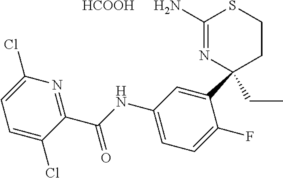

The coupling of [(S)-4-(5-amino-2-fluoro-phenyl)-4-ethyl-5,6-dihydro-4H-[1,3]thiazin-2-yl]-carbamic acid tert-butyl ester and 3,6-dichloro-pyridine-2-carboxylic acid followed by deprotection of the intermediate yielded the title compound (32 mg) as a colorless solid. MS (ESI): m/z=427.1 [M+H]⁺.

### Example 12

### 6-Chloro-3-trifluoromethyl-pyridine-2-carboxylic acid [3-((S)-2-amino-4-ethyl-5,6-dihydro-4H-[1,3]thiazin-4-yl)-4-fluoro-phenyl]-amide; salt with formic acid

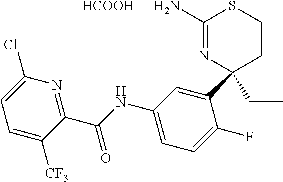

The coupling of [(S)-4-(5-amino-2-fluoro-phenyl)-4-ethyl-5,6-dihydro-4H-[1,3]thiazin-2-yl]-carbamic acid tert-butyl ester and 6-chloro-3-trifluoromethyl-pyridine-2-carboxylic acid followed by deprotection of the intermediate yielded the title compound (35 mg) as a colorless solid. MS (ESI): m/z=461.2 [M+H]⁺.

### Example 13

### Isoquinoline-3-carboxylic acid [3-((S)-2-amino-4-ethyl-5,6-dihydro-4H-[1,3]thiazin-4-yl)-4-fluoro-phenyl]-amide; salt with formic acid

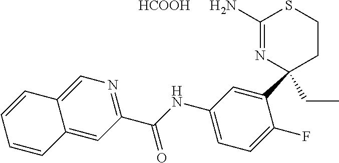

The coupling of [(S)-4-(5-amino-2-fluoro-phenyl)-4-ethyl-5,6-dihydro-4H-[1,3]thiazin-2-yl]-carbamic acid tert-butyl ester and isoquinoline-3-carboxylic acid followed by deprotection of the intermediate yielded the title compound (40 mg) as a colorless solid. MS (ESI): m/z=409.3 [M+H]⁺.

### Example 14

### Thieno[2,3-c]pyridine-7-carboxylic acid [3-((S)-2-amino-4-ethyl-5,6-dihydro-4H-[1,3]thiazin-4-yl)-4-fluoro-phenyl]-amide; salt with formic acid

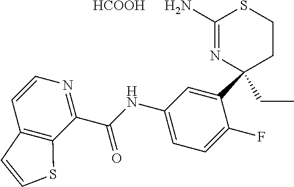

The coupling of [(S)-4-(5-amino-2-fluoro-phenyl)-4-ethyl-5,6-dihydro-4H-[1,3]thiazin-2-yl]-carbamic acid tert-butyl ester and thieno[2,3-c]pyridine-7-carboxylic acid (preparation described in Frohn, M. et al., Bioorg. & Med. Chem. Lett., 2008, 18, 5023) followed by deprotection of the intermediate yielded the title compound (41 mg) as a colorless solid. MS (ESI): m/z=415.2 [M+H]⁺.

### Example 15

### Benzo[b]thiophene-2-carboxylic acid [3-((S)-2-amino-4-ethyl-5,6-dihydro-4H-[1,3]thiazin-4-yl)-4-fluoro-phenyl]-amide; salt with formic acid

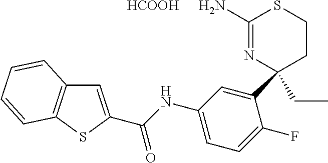

The coupling of [(S)-4-(5-amino-2-fluoro-phenyl)-4-ethyl-5,6-dihydro-4H-[1,3]thiazin-2-yl]-carbamic acid tert-butyl ester and benzo[b]thiophene-2-carboxylic acid followed by deprotection of the intermediate yielded the title compound (48 mg) as a colorless solid. MS (ESI): m/z=414.2 [M+H]⁺.

### Example 16

### 5-Methyl-thiophene-2-carboxylic acid [3-((S)-2-amino-4-ethyl-5,6-dihydro-4H-[1,3]thiazin-4-yl)-4-fluoro-phenyl]-amide; salt with formic acid

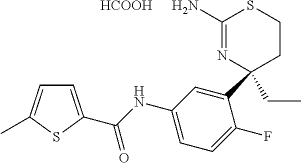

The coupling of [(S)-4-(5-amino-2-fluoro-phenyl)-4-ethyl-5,6-dihydro-4H-[1,3]thiazin-2-yl]-carbamic acid tert-butyl ester and 4-methyl-thiophene-2-carboxylic acid followed by deprotection of the intermediate yielded the title compound (22 mg) as a colorless solid. MS (ESI): m/z=378.3 [M+H]⁺.

### Example 17

### 1-Methyl-1H-pyrazole-3-carboxylic acid [3-((S)-2-amino-4-ethyl-5,6-dihydro-4H-[1,3]thiazin-4-yl)-4-fluoro-phenyl]-amide; salt with formic acid

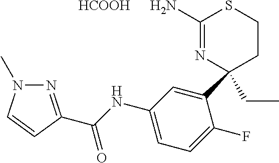

The coupling of [(S)-4-(5-amino-2-fluoro-phenyl)-4-ethyl-5,6-dihydro-4H-[1,3]thiazin-2-yl]-carbamic acid tert-butyl ester and 1-methyl-1H-pyrazole-3-carboxylic acid followed by deprotection of the intermediate yielded the title compound (27 mg) as a pale yellow solid. MS (ESI): m/z=362.3 [M+H]⁺.

### Example 18

### 2-Methyl-oxazole-4-carboxylic acid [3-((S)-2-amino-4-ethyl-5,6-dihydro-4H-[1,3]thiazin-4-yl)-4-fluoro-phenyl]-amide; salt with formic acid

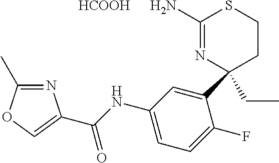

The coupling of [(S)-4-(5-amino-2-fluoro-phenyl)-4-ethyl-5,6-dihydro-4H-[1,3]thiazin-2-yl]-carbamic acid tert-butyl ester and 2-methyl-oxazole-4-carboxylic acid followed by deprotection of the intermediate yielded the title compound (21 mg) as a pale yellow solid. MS (ESI): m/z=363.3 [M+H]⁺.

### Example 19

### 2-Methyl-thiazole-4-carboxylic acid [3-((S)-2-amino-4-ethyl-5,6-dihydro-4H-[1,3]thiazin-4-yl)-4-fluoro-phenyl]-amide; salt with formic acid

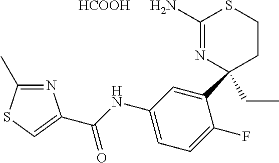

The coupling of [(S)-4-(5-amino-2-fluoro-phenyl)-4-ethyl-5,6-dihydro-4H-[1,3]thiazin-2-yl]-carbamic acid tert-butyl ester and 2-methyl-thiazole-4-carboxylic acid followed by deprotection of the intermediate yielded the title compound (29 mg) as a colorless solid. MS (ESI): m/z=379.3 [M+H]⁺.

### Example 20

The following test was carried out in order to determine the activity of the compounds of formula I:

### Immunofluorescence Resonance Energy Transfer (FRET) Assay for BACE2 Inhibition

BACE2 enzyme ectodomain (derived from plasmid “pET17b-T7-hu proBACE2”) was prepared as described in Ostermann et al., “Crystal Structure of Human BACE2 in Complex with a Hydroxyethylamine Transition-state Inhibitor”, Journal of Molecular Biology 2006, 355, 249-261. The pro-enzyme was stored at 4° C. at a concentration of 70 μg/ml.

The FRET assay was performed essentially as described in Grüninger-Leitch et al., Journal of Biological Chemistry (2002) 277(7) 4687-93 (“Substrate and inhibitor profile of BACE (beta-secretase) and comparison with other mammalian aspartic proteases”). In summary, a peptide is designed that is cleaved by the protease. The peptide is labelled with dabcyl at the N terminus and Lucifer Yellow at the C-terminus, such that for an intact peptide the Lucifer Yellow fluorescence is quenched by the dabcyl. When the peptide is cut by BACE2, the quenching is removed and a fluorescent signal is generated.

The assay was performed as described in Grueninger et al. 2002 at pH 4.5 using a substrate concentration of 5 μM. A FRET peptide based on the TMEM27 sequence was devised. dabcyl-QTLEFLKIPS-LucY. BACE2 had a high activity against this sequence, which is unrelated to the known APP-based substrates. Conversely, BACE1 had insignificant activity against this peptide.

The assay readout is the initial rate of change of fluorescence intensity giving a relative measure of BACE2 activity. Small values correspond to high inhibition and larger values to low inhibition. To determine IC₅₀ values (i.e. the concentration inhibiting the enzyme activity by 50%) of the compound for BACE2, typically, 12 assays were made with a range of concentrations chosen empirically to give low, high and intermediate inhibition of the protease. IC₅₀ values were determined using these assay values generated for a range of inhibitor concentrations and the curve fitting software XLfit (IDBS) using the Sigmoidal Dose-Response Model.

The preferred compounds according to formula I have an inhibitory activity in the above assay (IC₅₀) preferably of 5 nM to 50 μM, more preferably of 5 nM to 1 μM.

For example, the following compounds showed the following IC₅₀ values in the assay described above:

TABLE 1

| | IC₅₀ (BACE2) |
|---|---|
| Example | [nM] |
| 1 | 9 |
| 2 | 8 |
| 3 | 1031 |
| 4 | 2697 |
| 5 | 1001 |
| 6 | 440 |
| 7 | 5459 |
| 8 | 4927 |
| 9 | 41035 |
| 10 | 50924 |
| 11 | 23045 |
| 12 | 33490 |
| 13 | 9096 |
| 14 | 3080 |
| 15 | 916 |
| 16 | 667 |
| 17 | 2143 |
| 18 | 475 |
| 19 | 33577 |

### Example 21

### Detection of BACE2 Inhibition by Measuring TMEM27 Cleavage in Isolated Human Pancreatic Islets

Freshly isolated human islets from two different donors (male, 51 years, BMI: 27.5 kg/m2; female, 62 years, BMI: 22.2 kg/m2; circa 3000 islets per donor) were obtained from Dr. D. Bosco (Cell Isolation and Transplantation Center, Department of Surgery, Geneva, Switzerland) and maintained in CMRL-1066 (Invitrogen) at 5.6 mmol/l glucose supplemented with 10% FCS, 100 U/ml penicillin, 100 μg/ml streptomycin and 100 μg/ml gentamycin (Sigma) for 2 days before experiments. The present investigation was approved by the institutional ethics committee. Handpicked islets were cultured in the presence or absence of 200 nM of the compound of Example 1 for 72 h. Islets were collected by centrifugation and total proteins were extracted using CELYA lysis buffer CLB1 (Cat*9000, Zeptosens) following the manufacturer's protocol.

Total islet proteins (10 μg) were fractionated by NuPAGE 4-12% Bis-TrisGel (Cat*NP0321Box, Invitrogen) and transferred to nitrocellulose using iBlot system (Cat*IB3010-01, Invitrogen). The immunoblotting was performed with primary antibodies: mouse anti-TMEM27 monoclonal antibody (Roche Clone-3/3, 1 μg/ml); mouse anti-BACE2 monoclonal antibody (Roche Clone-1/9, 1 μg/ml); rabbit anti-GAPDH monoclonal antibody (Cat*2118, Cell Signaling, 1:4,000 dilution), followed by HRP-conjugated anti-mouse or anti-rabbit secondary antibodies (Pierce) using enhanced chemiluminescence for detection (Pierce).

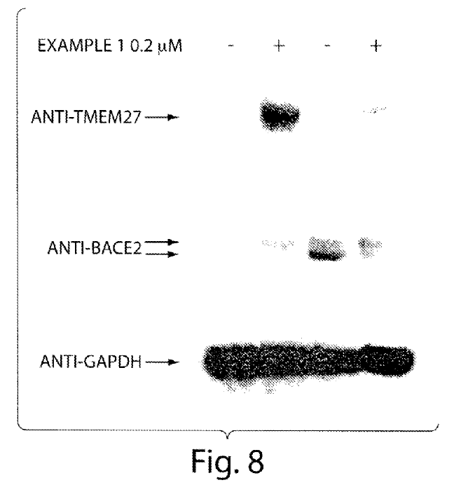

The Western blot (FIG. 8) shows that the compound of Example 1 stabilized the full length of TMEM27 as detected by mouse anti-hTMEM27 monoclonal antibody recognizing the C-terminus (Roche clone 3/3). hTMEM27 corresponds to the human sequence of TMEM27. The BACE2 inhibition resulted in the shift from the mature BACE2 (the lower band) to pro-BACE2 (the upper band) as recognized by mouse anti-hBACE2 (1/9) monoclonal antibody. Similar to other aspartic proteases, BACE2 is expressed as an inactive zymogen requiring the cleavage of its pro-sequence during the maturation process. pro-BACE2 requires autocatalytic pro-domain processing for enzymatic activation. Inhibition of BACE catalytic activity by the compound of Example 1 lead to a reduction of mature BACE2 and to an increase in the pro-BACE2. The inhibition of BACE2 activity also underlies the mechanism for the increase and stabilization in full length TMEM27 in human islets.

### Example 22

### Metabolic effects of the compound of Example 1 (5-chloro-pyridine-2-carboxylic acid [3-((S)-2-amino-4-methyl-5,6-dihydro-4H-[1,3]thiazin-4-yl)-4-fluoro-phenyl]-amide) in Zucker Diabetic Fatty (ZDF) rats

This study was conducted with male ZDF rats [ZDF/gmiCrl fa/fa] and lean rats [ZDF/gmiCrl fa/+] (Charles River Laboratories, Sulzfeld, Germany). The ZDF rats are a commonly used model of human type 2 diabetes characterized by insulin resistance, β-cell defects and hyperglycemia. Onset of diabetes in males is at the age of 8 to 10 weeks when fed a diabetogenic diet. All rats (ZDF and Lean rats) with an age of 6 weeks at the beginning of the experiment are fed a special diet (“PURINA PMI 5008”, ZDF_diet=Ssniff R/M−H) and housed 1 per cage (type 3). Ambient temperature is approximately 21° C. and relative humidity 55-65%. A 12 hours light-dark cycle is maintained in the rooms with all tests being performed during the light phase. Access to food and tap water is ad libitum.

6 week old ZDF rats were randomized to receive one of five treatments administered by oral gavage (except for the treatment with Liraglutide which had to be administered by subcutaneous injection):

Group 1 received Gelatin as Vehicle (n=11).

Group 2 received the compound of Example 1 at 0.2 mg/kg, daily (n=11). The dose is calculated to induce about 50% BACE2 inhibition after 24 hours.

Group 3 received the compound of Example 1 at 5 mg/kg, daily (n=11). Based on BACE2 inhibition activity this is the calculated dose for maximum effect.

Group 4 received the compound of Example 1 at 30 mg/kg, daily (n=11). At this dose, BACE2 activity should be totally blocked.

Group 5 received Liraglutide at 0.4 mg/kg, s.c daily (n=11).

The control group of lean rats (n=11) received vehicle.

TABLE 2
Treatment Groups

| | | | | Dose of | | | |
|---|---|---|---|---|---|---|---|
| | | Dose | | microsuspension | Number of | | Appl./ |
| | Treatment | (mg/kg) | | in gelatin (mg/ml) | ZDF rats | ROA | time |
| 1 | Vehicle: gelatin | — | | 2 ml/kg | 11 | p.o. | 4 p.m. |
| 2 | Example 1 | 0.2 | mg/kg | 2 ml/kg | 11 | p.o. | 4 p.m. |
| 3 | Example 1 | 5 | mg/kg | 2 ml/kg | 11 | p.o. | 4 p.m. |
| 4 | Example 1 | 30 | mg/kg | 2 ml/kg | 11 | p.o. | 4 p.m. |
| 5 | Liraglutide | 0.4 | mg/kg | 1 ml/kg | 11 | s.c. | 4 p.m. |

| | | | | | | |
|---|---|---|---|---|---|---|
| 6 | Lean ZDF rats | — | — | 11 | — | — |

Body weight and food intake were monitored daily. Blood glucose was measured weekly in all rats. After 3 weeks of treatment an oGTT was performed on 6 overnight fasted rats per groups. At about week 4 and after anesthesia, 2 to 3 ZDF rats per day and per group underwent pancreas surgery and in-situ pancreas perfusion with low/high glucose conditions. 120 eluted fractions per rat were collected for glucose and insulin quantification.

Oral Glucose Tolerance Test (oGTT)

An oGTT was conducted on day 18 of treatment. After on overnight fast of approximately 16 h post treatment, rats were given a glucose load of 2 g/kg by gavage. Blood samples were collected immediately prior to glucose challenge (0 min) and +10, +30, +60 and +120 min after glucose challenge and blood glucose and other plasma parameters were determined.

Blood glucose was measured with a blood glucose monitoring system (Accu-Chek Aviva, Roche Diagnostics GmbH, Rotkreuz, Switzerland). Insulin was measured by ELISA, using the Mercodia Rat Insulin ELISA (Mercodia AB, Uppsala, Sweden).

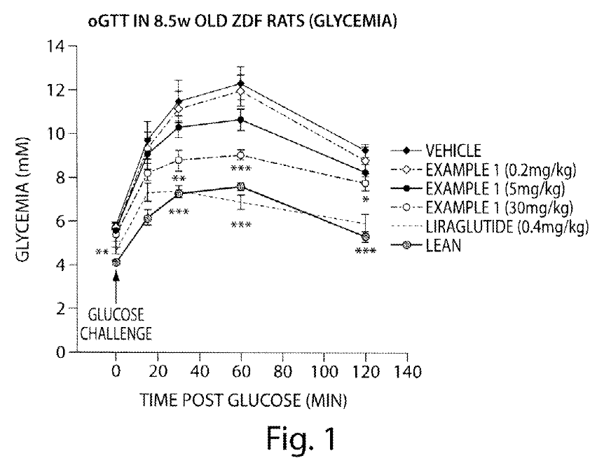

The results are shown in FIG. 1. Data were analyzed using the software SAS/JMP for Windows (version 6.0.0, SAS Institute Inc., Cary, N.C.). Data were expressed as mean±SEM (standard error of the mean). The number of rats was 6 per group. Comparison was made versus Vehicle, using Analysis of Variance ANOVA followed by post hoc Dunnett's test.

The Vehicle group is characterized by modestly elevated fasting blood glucose levels at time 0 (approximately 6 mM), followed by elevated and sustained glucose excursion recorded after oral glucose challenge indicating severe glucose intolerance in ZDF rats at this age.

Treatment with the compound of Example 1 dose-dependently reduced glucose area under the curve (AUC 0-120 minutes). Improvement of glucose tolerance by the compound of Example 1 (30 mg/kg) reached significance at 30′, 60′ and 120′ post glucose challenge compared to Vehicle. Treatment with the compound of Example 1 induced chronic efficacy in reducing overall post challenge glucose AUC. Liraglutide, a marketed drug for Type 2 Diabetes treatment, was used as positive control. Efficacy of the compound of Example 1 (30 mg/kg) was close to that induced by chronic treatment with Liraglutide (0.4 mg/kg).

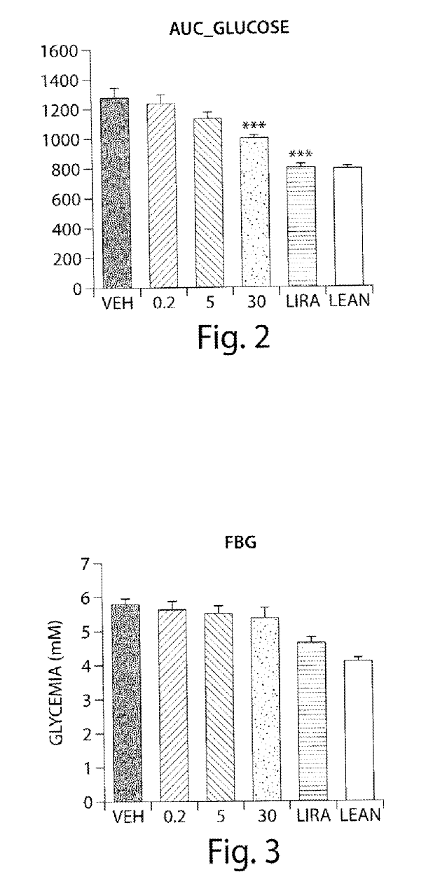

The quantification of glucose excursions during oGTT in 8.5 week old ZDF rats treated for 17 days with either vehicle, the compound of Example 1 or Liraglutide is further illustrated in FIG. 2. AUC stands for Area Under the Curve (0-120 minutes). Units are mM*min. AUC were calculated by the trapezoidal integration rule. This calculation stands for time 0 to 120 min post glucose challenge.

Chronic treatment with the compound of Example 1 induced dose dependent reduction of glucose AUC reaching significant values at 30 mg/kg (***p<0.001 compared to Vehicle, ANOVA followed by Post hoc Dunnett's test). Data were expressed as mean±SEM.

The effect of chronic treatment with the compound of Example 1 on fasting blood glucose (FBG) of 8.5 week old ZDF rats is shown in FIG. 3. Chronic treatment with the compound of Example 1 (0.2-5-30 mg/kg) showed a trend to reduce fasting blood glucose after overnight fasting conditions (FBG) without reaching significance. Similarly, Liraglutide showed a statistically non significant tendency towards FBG reduction. As expected, lean rats are characterized by lower FBG compared to age-matched ZDF-vehicle treated rats. Data were expressed as mean±SEM. Comparison was made versus Vehicle, using ANOVA followed by post hoc Dunnett's test.

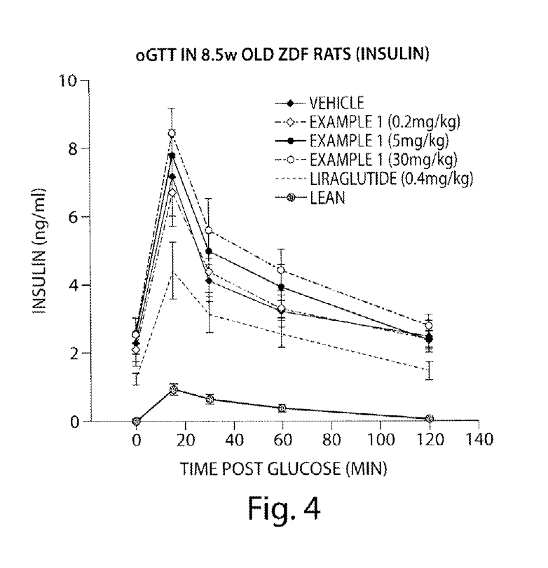

The Insulin levels during oGTT in 8.5 week old ZDF rats treated for 17 days with either vehicle, the compound of Example 1 or Liraglutide are shown in FIG. 4. ZDF rats were challenged with glucose (2 g/kg) at time 0: Vehicle-treated ZDF rats were characterized by rapid and pronounced increase in insulin following glucose challenge. Chronic treatment with the compound of Example 1 induced dose-dependent increase in insulin levels secreted during oGTT. Increase was mainly observed at peak of insulin secretion. Treatment with the compound of Example 1 did not change fasting insulin levels compared to vehicle. Chronic treatment with Liraglutide decreased both fasting and post-challenge insulin levels. Data were expressed as mean±SEM. Comparison was made between groups treated with the compound of Example 1 versus Vehicle, using ANOVA followed by post hoc Dunnett's test.

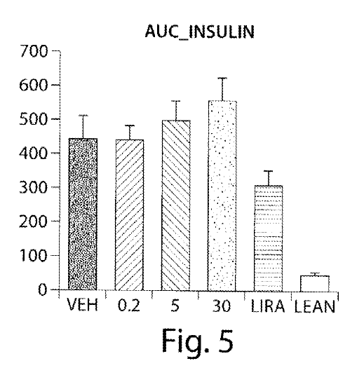

The Insulin AUC (0-120 minutes) during oGTT in 8.5 week old ZDF rats treated for 17 days with either vehicle, the compound of Example 1 or Liraglutide is further illustrated in FIG. 5. AUC stands for Area Under the Curve. Y Units are ng/ml*min. AUC were calculated by the trapezoidal rule. This calculation is done for time 0 to 120 min post glucose challenge. Chronic treatment with the compound of Example 1 induced dose-dependent increase in insulin levels without reaching significance. Data were expressed as mean±SEM.

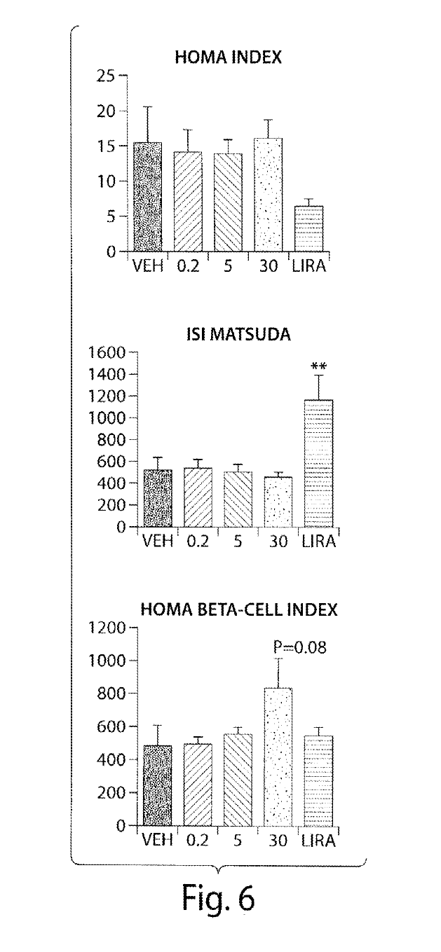

In addition, the HOMA_IR, the ISI Matsuda and HOMA β-cell Indices were calculated from the data measured in 8.5 week old ZDF rats after 17 days of treatment with either Vehicle, the compound of Example 1 or Liraglutide. The data are illustrated in FIG. 6 and were expressed as mean±SEM (N=6 per group). Comparison was made between groups treated with the compound of Example 1 versus Vehicle, using ANOVA followed by post hoc Dunnett's test. Chronic treatment with the compound of Example 1 did not impact on hepatic (HOMA) or whole body insulin resistance (MATSUDA) indices. In contrast, the compound of Example 1 dose-dependently improved HOMA-β insulin resistance index. This suggests that the compound of Example 1 is improving islet and β-cell function.

### Assessment of β-Cell Function by In Situ Pancreas Perfusion

Rats were anesthetized (Temgesic (0.1 ml/100 g) first, then anesthetic cocktail: Ketamine (77 mg/kg), Xylazine (11 mg/kg), i.p. injection, volume 2 ml/kg). Pancreas was surgically isolated from other connecting organs and from nerves and veins and artery afferences and efferences, keeping access to abdominal aorta and portal vein which are both cannulated. Once surgery was done, rats were placed into a temperature controlled box (37° C.) and pancreata were connected to infusion pumps via abdominal aorta.

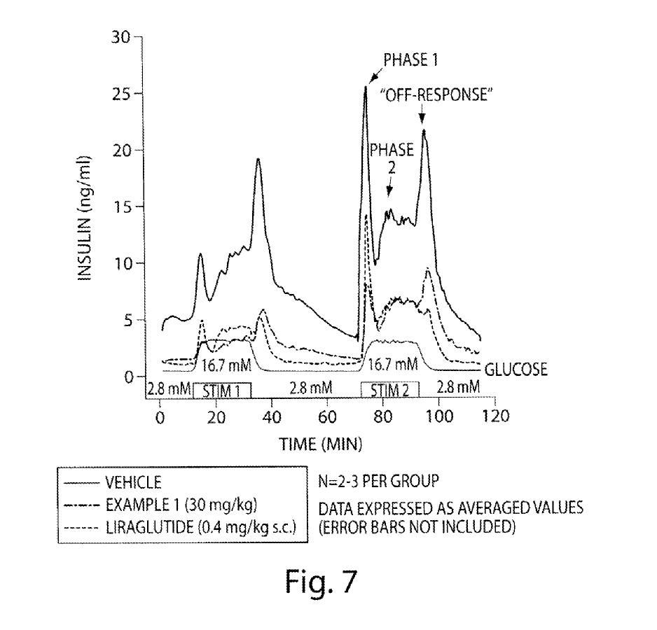

The glucose-stimulated insulin secretion (GSIS) was obtained by perfusing pancreata with Krebs-Ringer buffer containing low/high glucose concentration as described into protocol designed in FIG. 7. Basically, pancreata were first perfused with freshly prepared Krebs-Ringer solution (5 ml/min) containing low glucose concentration (2.8 mM) for about 30 minutes, stabilizing basal insulin secretion. Then, the first stimulation with high glucose concentration solution (16.7 mM) was given to sensitize the pancreata, leading to modest phase 1 and phase 2 insulin secretion. Finally, the second stimulation of pancreata with high glucose concentration (16.7 mM) at about 75 minutes led to full insulin secretion as demonstrated by rapid and elevated phase 1 followed by sustained and long-lasting phase 2 and “off-response” (see curve of Vehicle in FIG. 7). Treatment with the compound of Example 1 (30 mg/kg) reduced basal insulin secretion and AUC off-response compared to vehicle. The compound of Example 1 normalized the insulin secretion profile (Phase 1/Phase 2) and therefore prevents hyperinsulinemia.

Pancreas elution fractions were collected in 96-well plates (via a catheter introduced into the portal vein) at regular time intervals and immediately cooled down to 4° C. and subsequently stored at −20° C. until analyzed. At least, 120 eluted fractions per rat were collected for measurement of glucose and insulin levels.

### Example A

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula I | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxyde (yellow) | 0.8 | mg | 1.6 | mg |
| Titan dioxide | 0.8 | mg | 1.6 | mg |

The active ingredient is sieved and mixed with microcrystalline cellulose and the mixture is granulated with a solution of polyvinylpyrrolidone in water. The granulate is mixed with sodium starch glycolate and magesiumstearate and compressed to yield kernels of 120 or 350 mg respectively. The kernels are lacquered with an aqueous solution/suspension of the above mentioned film coat.

### Example B

Capsules containing the following ingredients can be manufactured in a conventional manner:

| Ingredients | Per capsule | |
|---|---|---|
| Compound of formula I | 25.0 | mg |
| Lactose | 150.0 | mg |
| Maize starch | 20.0 | mg |
| Talc | 5.0 | mg |

The components are sieved and mixed and filled into capsules of size 2.

### Example C

Injection solutions can have the following composition:

| Compound of formula I | 3.0 | mg |
|---|---|---|
| Polyethylene Glycol 400 | 150.0 | mg |
| Acetic Acid | q.s. ad pH 5.0 | |
| Water for injection solutions | ad 1.0 | ml |

The active ingredient is dissolved in a mixture of Polyethylene Glycol 400 and water for injection (part). The pH is adjusted to 5.0 by Acetic Acid. The volume is adjusted to 1.0 ml by addition of the residual amount of water. The solution is filtered, filled into vials using an appropriate overage and sterilized.

### Example D

Soft gelatin capsules containing the following ingredients can be manufactured in a conventional manner:

| Capsule contents | | |
|---|---|---|
| Compound of formula I | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titan dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example E

Sachets containing the following ingredients can be manufactured in a conventional manner:

| | | |
|---|---|---|
| Compound of formula I | 50.0 | mg |
| Lactose, fine powder | 1015.0 | mg |
| Microcristalline cellulose (AVICEL PH 102) | 1400.0 | mg |
| Sodium carboxymethyl cellulose | 14.0 | mg |
| Polyvinylpyrrolidon K 30 | 10.0 | mg |
| Magnesiumstearate | 10.0 | mg |
| Flavoring additives | 1.0 | mg |

The active ingredient is mixed with lactose, microcristalline cellulose and sodium carboxymethyl cellulose and granulated with a mixture of polyvinylpyrrolidone in water. The granulate is mixed with magnesiumstearate and the flavouring additives and filled into sachets.

1. A compound of formula Ia, 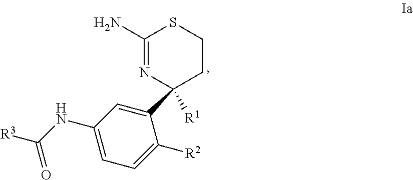
wherein
R¹ is ethyl;
R² is selected from the group consisting of C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

2. A compound according to claim 1, wherein R² is halogen.

3. A compound according to claim 1, wherein R² is fluoro.

4. A compound according to claim 1, wherein R³ is heteroaryl, said heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl.

5. A compound according to claim 1, wherein R³ is heteroaryl selected from the group consisting of thienyl, oxazolyl, thiazolyl, pyrazolyl, pyridyl, pyrimidinyl, pyrazinyl, isoquinolinyl, thieno[2,3-c]pyridyl and benzo[b]thienyl, said heteroaryl being unsubstituted or substituted by one, two or three groups selected from the group consisting of C₁₋₇-alkyl, halogen, halogen-C₁₋₇-alkyl and phenyl.

6. A compound according to claim 1, wherein R³ is phenyl, said phenyl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl and phenyl.

7. A compound according to claim 1, selected from the group consisting of
5-chloro-pyridine-2-carboxylic acid [3-((S)-2-amino-4-ethyl-5,6-dihydro-4H-[1,3]thiazin-4-yl)-4-fluoro-phenyl]-amide,
pyridine-2-carboxylic acid [3-((S)-2-amino-4-ethyl-5,6-dihydro-4H-[1,3]thiazin-4-yl)-4-fluoro-phenyl]-amide,
N-[3-((S)-2-amino-4-ethyl-5,6-dihydro-4H-[1,3]thiazin-4-yl)-4-fluoro-phenyl]-4-chloro-benzamide,
5-chloro-pyrazine-2-carboxylic acid [3-((S)-2-amino-4-ethyl-5,6-dihydro-4H-[1,3]thiazin-4-yl)-4-fluoro-phenyl]-amide,
5-chloro-pyrimidine-2-carboxylic acid [3-((S)-2-amino-4-ethyl-5,6-dihydro-4H-[1,3]thiazin-4-yl)-4-fluoro-phenyl]-amide,
3-trifluoromethyl-pyridine-2-carboxylic acid [3-((S)-2-amino-4-ethyl-5,6-dihydro-4H-[1,3]thiazin-4-yl)-4-fluoro-phenyl]-amide,
3-phenyl-pyridine-2-carboxylic acid [3-((S)-2-amino-4-ethyl-5,6-dihydro-4H-[1,3]thiazin-4-yl)-4-fluoro-phenyl]-amide,
4-chloro-pyridine-2-carboxylic acid [3-((S)-2-amino-4-ethyl-5,6-dihydro-4H-[1,3]thiazin-4-yl)-4-fluoro-phenyl]-amide,
6-methyl-pyridine-2-carboxylic acid [3-((S)-2-amino-4-ethyl-5,6-dihydro-4H-[1,3]thiazin-4-yl)-4-fluoro-phenyl]-amide,
3,6-dichloro-pyridine-2-carboxylic acid [3-((S)-2-amino-4-ethyl-5,6-dihydro-4H-[1,3]thiazin-4-yl)-4-fluoro-phenyl]-amide,
6-chloro-3-trifluoromethyl-pyridine-2-carboxylic acid [3-((S)-2-amino-4-ethyl-5,6-dihydro-4H-[1,3]thiazin-4-yl)-4-fluoro-phenyl]-amide,
isoquinoline-3-carboxylic acid [3-((S)-2-amino-4-ethyl-5,6-dihydro-4H-[1,3]thiazin-4-yl)-4-fluoro-phenyl]-amide,
thieno[2,3-c]pyridine-7-carboxylic acid [3-((S)-2-amino-4-ethyl-5,6-dihydro-4H-[1,3]thiazin-4-yl)-4-fluoro-phenyl]-amide,
benzo[b]thiophene-2-carboxylic acid [3-((S)-2-amino-4-ethyl-5,6-dihydro-4H-[1,3]thiazin-4-yl)-4-fluoro-phenyl]-amide,
5-methyl-thiophene-2-carboxylic acid [3-((S)-2-amino-4-ethyl-5,6-dihydro-4H-[1,3]thiazin-4-yl)-4-fluoro-phenyl]-amide,
1-methyl-1H-pyrazole-3-carboxylic acid [3-((S)-2-amino-4-ethyl-5,6-dihydro-4H-[1,3]thiazin-4-yl)-4-fluoro-phenyl]-amide,
2-methyl-oxazole-4-carboxylic acid [3-((S)-2-amino-4-ethyl-5,6-dihydro-4H-[1,3]thiazin-4-yl)-4-fluoro-phenyl]-amide, and
2-methyl-thiazole-4-carboxylic acid [3-((S)-2-amino-4-ethyl-5,6-dihydro-4H-[1,3]thiazin-4-yl)-4-fluoro-phenyl]-amide,
or pharmaceutically acceptable salts thereof.

8. A pharmaceutical composition comprising a compound of claim 1 and a pharmaceutically acceptable carrier and/or adjuvant.

9. A method for the treatment or prevention of diabetes, which method comprises administering, to a human being or animal in need thereof, a therapeutically active amount of a compound of formula I, 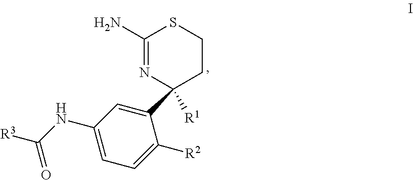
wherein
R¹ is C₁₋₇-alkyl or C₃₋₇-cycloalkyl;
R² is selected from the group consisting of hydrogen, C₁₋₇-alkyl, halogen, cyano and C₁₋₇-alkoxy; and
R³ is aryl or heteroaryl, said aryl or heteroaryl being unsubstituted or substituted by one, two or three groups selected from the group consisting of C₁₋₇-alkyl, halogen, halogen-C₁₋₇-alkyl, C₁₋₇-alkoxy, halogen-C₁₋₇-alkoxy, cyano, hydroxy-C₁₋₇-alkyl, oxo and phenyl;
or a pharmaceutically acceptable salt thereof.

10. A method according to claim 9 wherein:
R¹ is ethyl; and
R² is selected from the group consisting of C₁₋₇-alkyl, halogen, cyano and C₁₋₇-alkoxy.
